(19) United States
(12) Patent Application Publication
(10) Pub. No.: US 2025/0090502 A1
(43) Pub. Date: Mar. 20, 2025

(54) METHODS OF TREATING, AMELIORATING, AND/OR PREVENTING BACTERIAL INFECTION, OR METHODS OF REDUCING ADVERSE EFFECTS CAUSED BY BACTERIA

(71) Applicant: Drexel University, Philadelphia, PA (US)

(72) Inventors: Donald Carl Hall, Delta (PA); Hai-Feng Ji, Cherry Hill, NJ (US); Garth David Ehrlich, Philadelphia, PA (US)

(21) Appl. No.: 18/884,251

(22) Filed: Sep. 13, 2024

Related U.S. Application Data
(60) Provisional application No. 63/538,607, filed on Sep. 15, 2023.

(51) Int. Cl. A61K 31/4192; A61K 31/415; A61K 31/4166; A61K 31/4178; A61K 31/422; A61K 31/4245; A61K 31/433; A61K 31/702; A61P 31/04

(52) U.S. Cl. CPC A61K 31/4192; A61K 31/415; A61K 31/4166; A61K 31/4178; A61K 31/422; A61K 31/4245; A61K 31/433; A61K 31/702; A61P 31/04

(57) ABSTRACT

Described herein is a method of treating, ameliorating and/or preventing a bacterial infection in a subject in need thereof. The method includes administering to the subject an effective amount of a compound having the structure of

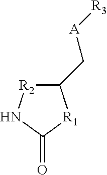

or

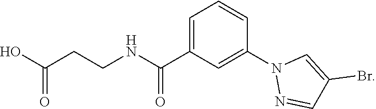 [SMILES: O=C(O)CCNC(=O)c1cccc(-n2cc(Br)cn2)c1]

Also described here is a method of reducing or eliminating an adverse effect caused by a bacterium in a subject. The method includes administering to the subject a compound of the disclosure. Also described are compositions and kits for performing the methods herein, which include a compound of the disclosure and an antibiotic.

### CROSS-REFERENCE TO RELATED APPLICATIONS

The present application claims priority under 35 U.S.C. § 119(e) to U.S. Provisional Patent Application No. 63/538,607, filed Sep. 15, 2023, which is incorporated herein by reference in its entirety.

### STATEMENT REGARDING FEDERALLY SPONSORED RESEARCH

This invention was made with government support under AI146177 awarded by the National Institute of Health. The government has certain rights in the invention.

### BACKGROUND

Antibiotics research has been focused on killing rapidly growing and dividing planktonic, single-celled bacteria. While this strategy has proven largely successful for the treatment of acute infections in the past, the widespread use of antibiotics has also caused development of genetic antibiotic resistance mechanisms in bacteria.

The virulence of pathogenic bacteria comes partially from the ability of the bacteria to release toxin, which causes lesions, hemolytic effects, and the like.

Therefore, there is a need for novel methods of treating bacterial infections, especially those caused by antibiotic-resistant bacteria. There is also a need for novel methods that reduce or eliminate certain adverse effects caused by the bacterial infections. The present invention addresses these needs.

### SUMMARY

In some aspects, the present invention is directed to the following non-limiting embodiments:

Method of Treating, Ameliorating, and/or Preventing a Bacterial Infection

In some aspects, the present invention is directed to a method of treating, ameliorating, and/or preventing a bacterial infection in a subject in need thereof.

In some embodiments, the method comprises administering to the subject an effective amount of at least one compound selected from:
(a) a compound of Formula I:

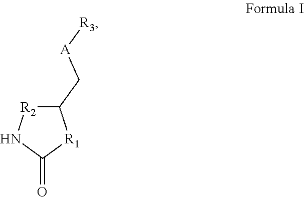

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

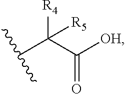

and
R₄ and R₅ are each independently H, halogen, C₁-C₆ alkyl, C₁-C₆ alkoxy, or —OH;
(b)

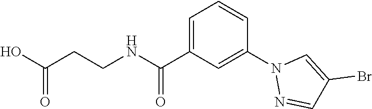 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

In some embodiments, in Formula I, A is

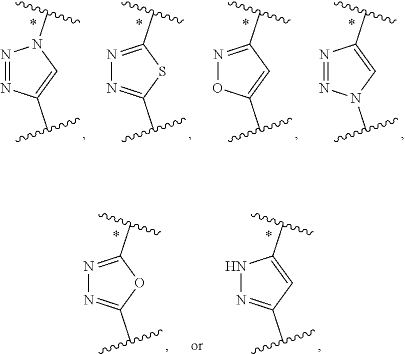 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, the compound of Formula I is at least one selected from the group consisting of:

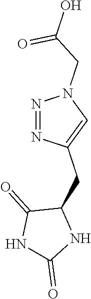 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

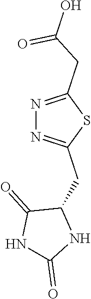 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C4-G2A);

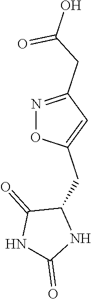 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

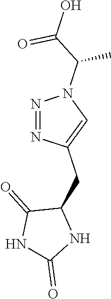 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C

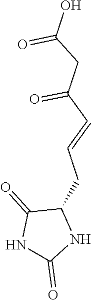 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

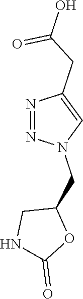 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

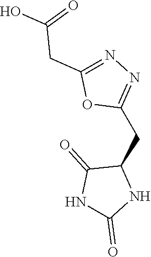 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

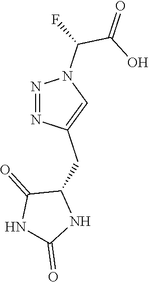 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

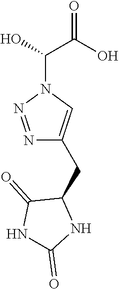 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

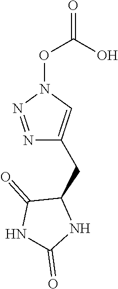 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

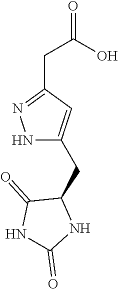 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

In some embodiments, the bacterial infection is caused by an antibiotic resistant bacterium.

In some embodiments, he antibiotic resistance of the bacterium is derived from and/or involves formation of a biofilm or development of a stringent response by the bacterium.

In some embodiments, the bacterium is a gram-positive bacterium or a gram-negative bacterium.

In some embodiments, the bacterium is at least one of B. burgdorferi, E. coli, H. influenzae, N. gonorrhoeae, P. aeruginosa, S. epidermidis, S. pneumoniae, and S. aureus.

In some embodiments, the administration of the compound inhibits or reverses biofilm formation, inhibits a stringent response, inhibits production of a toxin, inhibits a hemolytic effect, reduces ability to protect against an oxidative stress, reduces antibiotic resistance, reduces the acquisition of antibiotic resistance, reduces horizontal gene transfer, and/or reduce transformation in the bacterium causing the infection.

In some embodiments, the bacterial infection causes a skin lesion, and wherein the compound treats, ameliorates and/or prevents the skin lesion in the subject.

In some embodiments, the method further comprises administering to the subject an antibiotic effective for treating, ameliorating and/or preventing the bacterial infection.

In some embodiments, the subject is a mammal, optionally a human.

### Method of Reducing or Eliminating Adverse Effect Caused by Bacterium

In some aspects, the present invention is directed to a method of reducing or eliminating an adverse effect caused by a bacterium in a subject.

In some embodiments, the adverse effect is at least one selected from the group consisting of biofilm formation, toxin production, a hemolytic effect, and a skin lesion formation.

In some embodiments, the method comprises administering to the subject an effective amount of at least one compound selected from:
(a) a compound of Formula I:

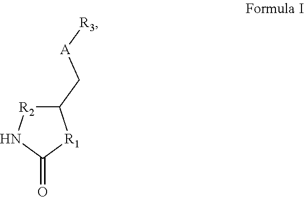

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

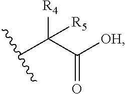

and
R₄ and R₅ are each independently H, halogen, C₁-C₆ alkyl, C₁-C₆ alkoxy, or —OH;
(b)

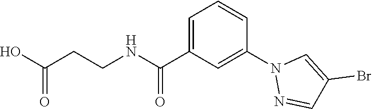 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

In some embodiments, in Formula I, A is

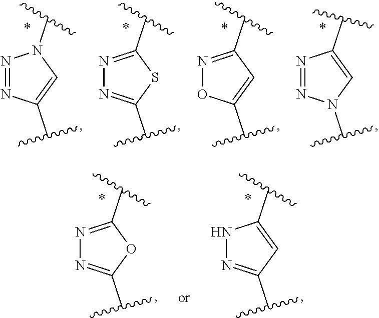 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, the compound of Formula I is at least one selected from the group consisting of:

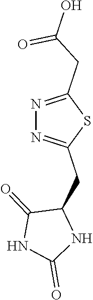 [SMILES: O=C(O)Cc1nnc(C[C@H]2NC(=O)NC2=O)s1]

(R)-2-(4-((2,5-dioxoimidazolidin-4-yl)methyl)-1H-1,2,3-triazol-1-yl)acetic acid (C14 or HEJ14);

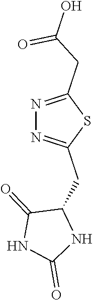 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

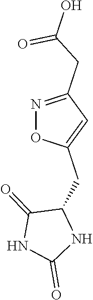 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

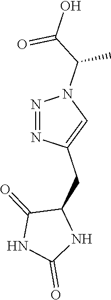 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

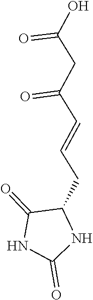 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

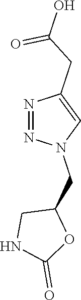 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

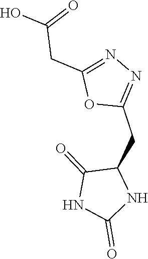 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

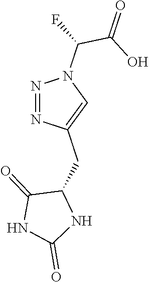 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

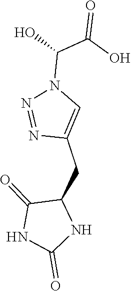 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

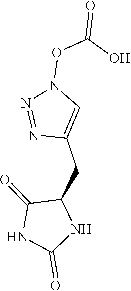 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

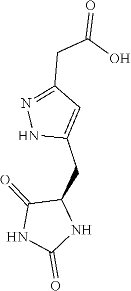 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

In some embodiments, the bacterium is an antibiotic-resistant bacterium.

In some embodiments, the antibiotic resistance of the bacterium is derived from and/or involves formation of a biofilm or development of a stringent response by the bacterium.

In some embodiments, the bacterium is a gram-positive bacterium or a gram-negative bacterium.

In some embodiments, the bacterium is at least one of B. burgdorferi, E. coli, H. influenzae, N. gonorrhoeae, P. aeruginosa, S. epidermidis, S. pneumoniae, and S. aureus.

In some embodiments, the method further comprises administering to the subject an antibiotic effective for killing or inhibiting growth of the bacterium.

In some embodiments, the subject is a mammal, optionally a human.

### Composition

In some aspects, the present invention is directed to a composition.

In some embodiments, the composition comprises:

at least one compound selected from:
(a) a compound of Formula I:

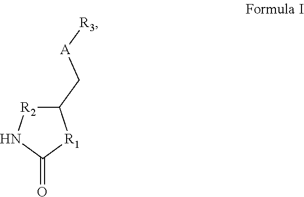

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃
   R₃ is —O—C(O)OH or

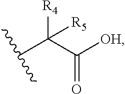

and
R₄ and R₅ are each independently H, halogen, C₁-C₆ alkyl, C₁-C₆ alkoxy, or —OH;
(b)

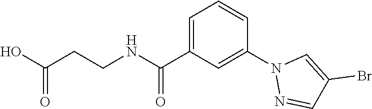 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof;
   and
   an antibiotic.

In some embodiments, in Formula I, A is

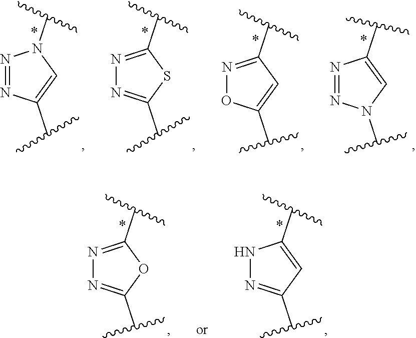 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen

In some embodiments, the compound of Formula I is at least one selected from the group consisting of:

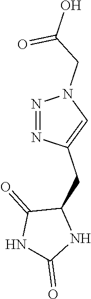 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

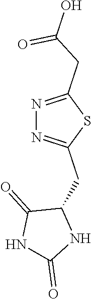 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

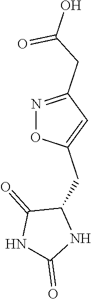 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

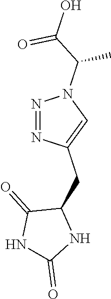 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

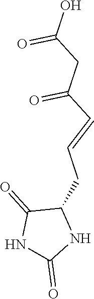 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

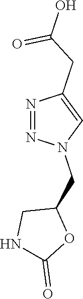 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

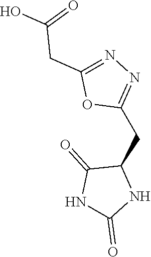 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

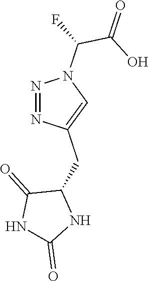 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

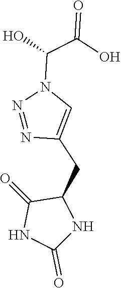 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

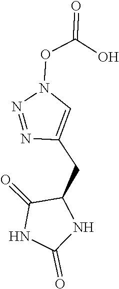 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

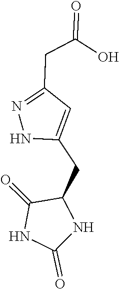 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

In some embodiments, the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

In some embodiments, the composition further comprises a pharmaceutically acceptable carrier.

### Kit

In some aspects, the present invention is directed to a kit.

In some embodiments, the kit comprises:

at least one compound selected from:
(a) a compound of Formula I:

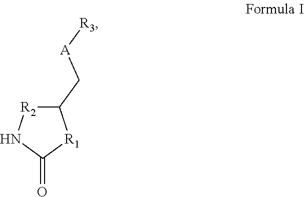

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

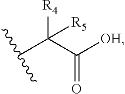

and
R₄ and R₅ are each independently H, halogen, C₁-C₆ alkyl, C₁-C₆ alkoxy, or —OH;
(b)

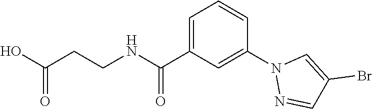 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof;
   and
   an antibiotic.

In some embodiments, in Formula I, A is

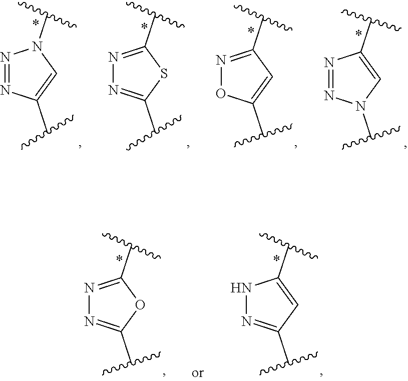 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen

In some embodiments, the compound of Formula I is at least one selected from the group consisting of:

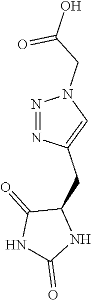 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

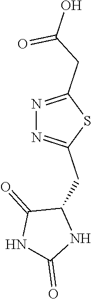 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

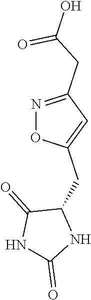 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

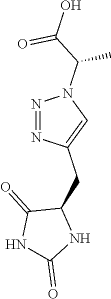 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

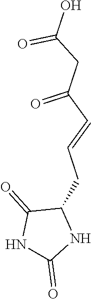 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

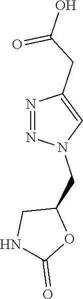 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

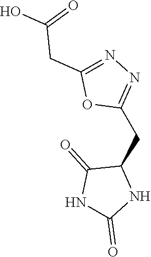 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

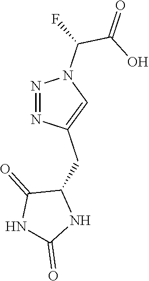 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

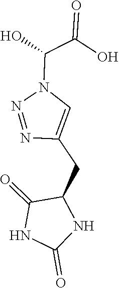 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

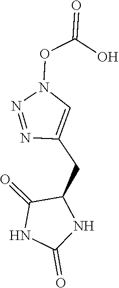 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

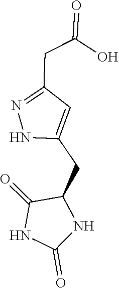 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

In some embodiments, the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

In some embodiments, the composition further comprises a manual instructing that the compound and the antibiotic are to be administered to a subject infected with a bacterium, optionally an antibiotic-resistant bacterium.

### Method of Reducing the Acquisition of Antibody Resistance in a Bacterium

In some aspects, the present invention is directed to a method of reducing the acquisition of antibody resistance in a bacterium.

In some embodiments, the method comprises contacting the bacterium with at least one compound selected from:
(a) a compound of Formula I:

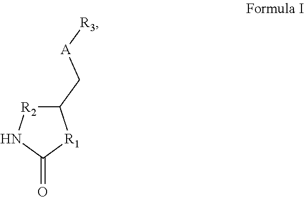

or a salt, solvate, tautomer, N-oxide, geometric isomer, stereoisomer thereof,
and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

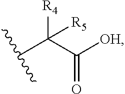

and
R₄ and R₅ are each independently H, halogen, C₁-C₆ alkyl, C₁-C₆ alkoxy, or —OH;
(b)

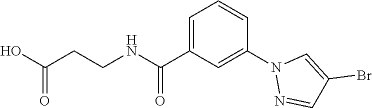 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

In some embodiments, in Formula I, A is

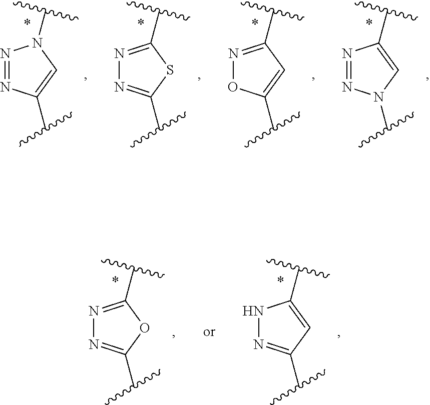 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, the compound of Formula I is at least one selected from the group consisting of:

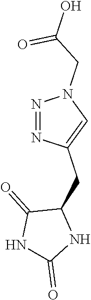 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

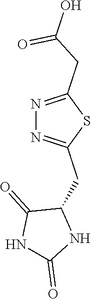 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

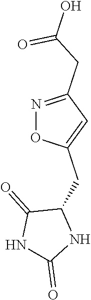 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

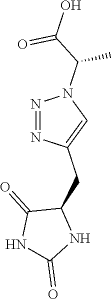 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

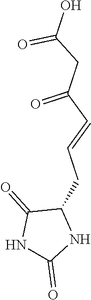 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

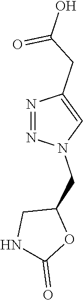 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

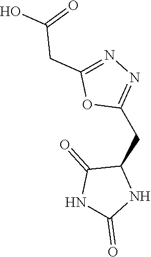 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

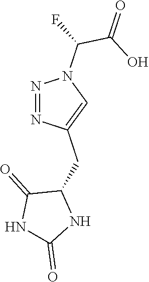 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

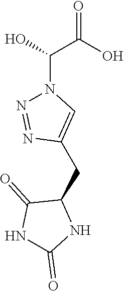 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

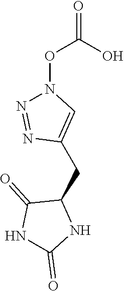 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

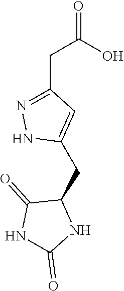 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

In some embodiments, the method further comprises contacting the bacterium with an antibiotic.

In some embodiments, the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

In some embodiments, the bacterium is at least one selected from the group consisting of B. burgdorferi, E. coli, H. influenzae, N. gonorrhoeae, P. aeruginosa, S. epidermidis, S. pneumoniae, and S. aureus.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 illustrate certain aspects of a pipeline for determination of effective RelA inhibitors, in accordance with some embodiments.

FIG. 2 shows the structure of RelA with HEJ14 (small circle) inhibitor docked in the binding pocket, in accordance with some embodiments. Large circle atomic structure of HEJ14.

FIGS. 3A-3D demonstrate the effectiveness of deleting or inhibition RelA as an anti-bacterial strategy, in accordance with some embodiments. FIG. 3A: transformation frequency of wild type (Rd) Haemophilus influenzae versus ΔRelA Rd; FIG. 3B: transformation frequency of Rd versus ΔRelA/ΔSpot Rd; FIG. 3C: planktonic treatments: no treatment (left); kanamycin treatment (middle); HEJ14+kanamycin treatment (right). Left=WT Rd; middle=ΔRelA Rd and right=ΔRelA/ΔSpot Rd. FIG. 3D: biofilm treatments: no treatment (left); kanamycin treatment (middle); HEJ14+kanamycin treatment (right). Left=WT Rd; middle=ΔRelA Rd and right=ΔRelA/ΔSpot Rd.

FIG. 4 illustrates certain aspects of the effect of HEJ14 on pyocyanin toxin production by the Gram-negative bacteria P. aeruginosa, in accordance with some embodiments. As shown in FIG. 4, the untreated P. aeruginosa culture (“Untreated”) displayed a deep green-blue color, indicating the presence of the toxin pyocyanin. The tube containing P. aeruginosa culture treated with HEJ14 (“HEJ14”) displayed a yellow color, indicating a decreased pyocyanin production. The bar chart illustrates the effect of HEJ14 on pyocyanin by P. aeruginosa at various HEJ14 concentrations.

FIGS. 5A-5B demonstrate that HEJ14 reduces toxin production of P. aeruginosa and protects wax worm larvae from death caused by P. aeruginosa infection, in accordance with some embodiments. The color of larvae in the dishes shows whether the larvae are alive or dead, wherein white color indicates the larvae is alive and brown color indicates the worm is dead.

FIG. 5A: HEJ14 reduced toxin production of P. aeruginosa. The label “DMSO 48 h” above the dish indicate that the larvae were injected with supernatant of P. aeruginosa culture grown with DMSO carrier only. The label “HEJ14 48 h” above the dish indicates that the larvae were injected with supernatants of P. aeruginosa culture grown with HEJ14. Kaplan Meier Plot showing increased survival of wax worms receiving supernatant from HEJ14 treated P. aeruginosa culture. FIG. 5B: HEJ14 protected wax worm larvae from the lethality caused by P. aeruginosa infection. In FIG. 5B, larvae were injected with either live P. aeruginosa (10⁵ CFU) and HEJ14, or the live bacteria and DMSO carrier only. Right dish: Larvae were injected with live P. aeruginosa (10⁵ CFU) & 50 μM HEJ14. The Kaplan Meier Plot showing increased survival of wax worms receiving HEJ14 treated P. aeruginosa.

FIGS. 6A-6B demonstrate that HEJ14 treatments of the Gram-positive bacteria Staphylococcus aureus resulted in increased stationary phase growth and suppressed agr expression, in accordance with some embodiments. Cultures of a S. aureus expressing an agr-GFP reporter were incubated with vehicle (0 μM) or various concentrations of HEJ14 (125 M, 250 μM, and 500 μM). The bacterial growth (OD600) and GFP signal intensity (RFU) were measured for a duration of 24 hours at 20-minute intervals. FIG. 6A: Bacterial growth (OD600).

FIG. 6B: agr expression as measured by GFP signal intensity (RFU) normalized to OD600.

FIG. 7 demonstrates that the supernatants from HEJ14-treated S. aureus cultures have decreased hemolytic activity as compared with S. aureus cultures treated by vehicle, in accordance with some embodiments. S. aureus broth cultures were incubated with vehicle or HEJ14 (125 μM) for 24 hrs. The supernatants were filter sterilized (0.2 μm) and 5 μL plated in serial dilutions on 5% sheep's blood agar, starting with V1 (vehicle treated plate) and R₁ (HEJ14 treated plate).

FIGS. 8A-8D demonstrate that HEJ14 protects animals from S. aureus infection, in accordance with some embodiments. The dorsal skin surfaces of anesthetized mice were shaved; a 100 μL volume of 3×10⁷ CFU of methicillin-resistant S. aureus (MRSA) (strain USA300 LAC::lux) was injected intradermally with, from left to right, vehicle, 125 μM of HEJ14, or 250 μM of HEJ14. FIG. 8A: Digital photographs of dorsal lesions. FIG. 8B: Digital bioluminescent signals of S. aureus. FIG. 8C: images from FIG. 8A were analyzed using Image J to obtain total lesion areas. FIG. 8D Images from FIG. 8B were analyzed by Image J to determine bacterial burden based on photon count. Significantly decreased lesion size and bacterial bioburden were observed in the HEJ14-treated mice, in a dose-dependent manner.

FIG. 9 demonstrates that HEJ14 eliminates bacterial aggregation and reduces biofilm formation in the Gram-negative bacteria Escherichia coli C, in accordance with some embodiments. Top panels: aggregation was observed at the bottom of the culture tube in untreated E. coli; it is completely absent in the tube containing E. coli C treated with HEJ14. Bottom panels: Micrographs of the aggregates (or the lack thereof in the case of HEJ14 treated sample).

FIGS. 10A-10B illustrate certain aspects of the effect of ampicillin concentration on planktonic growth and biofilm formation, in accordance with some embodiments. FIG. 10A: Growth of E. coli C cells in liquid culture at various concentrations of ampicillin (OD₆₀₀). FIG. 10B: Amount of biofilm formed at various concentrations of ampicillin. AMP #=ampicillin at #μg·mL⁻¹ concentration.

FIGS. 11A-11B demonstrate that anti-RelA compounds and ampicillin have synergistic effects on E. coli C biofilm volume and cell viability, in accordance with some embodiments.

FIG. 11A: Biofilm degradation utilizing hit compounds and ampicillin. FIG. 11B: AlamarBlue viability assay following combined treatment of cells with hit compounds and ampicillin shows a reduction in bacterial viability. A=S1-G1A (50 μM), B=S1-G1B (50 μM), Amp #=ampicillin at a concentration of #μg·mL⁻¹. SH=serine hydroxamate. IDR=IDR 1018. * Indicates statistical significance.

FIGS. 12A-12B demonstrate that S. aureus skin infection induces distinct global transcriptional responses, in accordance with some embodiments. S. aureus skin infection was performed on wild-type (WT) mice. Naive skin and day 3 infected skin were harvested on day 3 post-infection for RNA sequencing (n=5 per group). The false discovery rate (FDR)≤0.05 and the absolute value of log 2 fold change ≥1 (|log 2 FC|≥1) were used as the threshold to determine statistically significant differences in gene expression (PMID:12584122). Based on the genes that were significantly different in expression, DEGs were determined and PCA score plot and KEGG pathway analyses was performed using BGI Dr. Tom, a web-based tool for RNA data analysis and visualization. FIG. 12A: Principal component analysis (PCA) score plot. FIG. 12B: KEGG pathway of up-regulated genes in infected WT skin.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In a previous study (described in WO 2023/049796 A1, which is incorporated herein in its entirety by reference), a group of compounds that inhibit RelA and other RelA/SpoT Homology (RSH) enzymes in bacteria was described.

In the study described herein (“the present study”), a non-limiting example of the group of RelA/RSH inhibitors, HEJ14 (also referred to as “C14” in WO 2023/049796 A1) was tested for the ability to inhibit certain adverse effects caused by some bacteria, as well as to treat, ameliorate, and/or prevent bacterial infection. Since the RelA enzyme is part of the ribosomal machinery, like other ribosomal components, it is very highly conserved among bacteria. It is therefore expected that the anti-bacterial effects of the compounds herein work in the majority of pathogenic bacteria. Indeed, the present study demonstrated the efficacy of the compounds herein with multiple Gram-negative and Gram-positive species.

Accordingly, in some aspects, the present invention is directed to a method of treating, ameliorating, and/or preventing a bacterial infection in a subject in need thereof.

In some aspects, the present invention is directed to a method of reducing or eliminating an adverse effect caused by or involving bacterial infection.

Furthermore, the present study discovered that some non-limiting examples of the RelA/RSH inhibitors herein, when combined with antibiotics, were able to significantly increase the effectiveness of the antibiotics.

Accordingly, in some aspects, the present study is directed to a composition or a kit including the RelA/RSH inhibitors and an antibiotic.

Due to the near universal ability of bacteria to acquire genes via horizontal gene transfer (HGT), antibiotic resistance rapidly appears in almost all pathogens whether it evolved in situ or resulted from HGT from one of the thousands of other bacterial species that make up the microbiota of humans, farm animals and environmental microbes exposed to the antibiotic. The present study discovered that the compounds herein are able to significantly reduce transformation in bacteria, thereby reducing HGT and antibiotic resistance acquisition.

Accordingly, in some aspects, the present study is directed to a method, a composition, or a kit for reducing transformation, horizontal gene transfer, and/or antibiotic acquisition in bacteria.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” “About” as used herein when referring to a measurable value such as an amount, a temporal duration, and the like, is meant to encompass variations of ±20% or ±10%, in certain embodiments ±5%, in certain embodiments ±%, in certain embodiments ±0.1% from the specified value, as such variations are appropriate to perform the disclosed methods.

A “biofilm” as used herein refers to a structured consortium of bacteria embedded in a self-produced polymer matrix consisting of polysaccharide, protein and DNA (Hoiby et al., International Journal of Antimicrobial Agents 2010, 35 (4), 322-332). This matrix is called the EPS (extracellular polymeric substance). This EPS can protect the biofilm excreting bacteria colonies from outside stresses. The combination of lowered metabolic rate and upregulation of protective enzymes in biofilms collectively make biofilm infections antibiotic resistant.

A “stringent response” as used herein refers to a systematic reaction to one or more stress element/condition applied to a cell or group of cells. The stresses that are often associated with the stringent response include starvation of essential amino acids, heat shock, iron limitation, population density, and oxidative stress (associated with antibiotic treatment). This stringent response is pleiotropic and leads to antibiotic resistance and formation of bacteria biofilms.

The term “effective amount” of an agent as used herein, e.g., a pharmaceutical formulation, refers to an amount effective, at dosages and for periods of time necessary, to achieve a desired therapeutic or prophylactic result.

The term “individual” or “subject” or “patient” as used herein refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject or patient is a human.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

The term “pharmaceutically acceptable carrier” as used herein refers to an ingredient in a pharmaceutical formulation, other than an active ingredient, which is nontoxic to a subject. A pharmaceutically acceptable carrier includes, but is not limited to a buffer, excipient, stabilizer, or preservative.

The term “treatment,” “treat,” or “treating” as used herein refers to clinical intervention in an attempt to alter the natural course of the individual being treated and can be performed either for prophylaxis or during the course of clinical pathology. Desirable effects of treatment include, but are not limited to, preventing occurrence or recurrence of bacterial infection, alleviation of symptoms, diminishment of any direct or indirect pathological consequences of the bacterial infection, decreasing the rate of bacterial infection progression, amelioration or palliation of the bacterial infection state, and remission or improved prognosis. In some embodiments, the compounds (and the antibiotic in accordance with some embodiments) are used to delay development of a bacterial infection or to slow the progression of a bacterial infection.

Method of Treating, Ameliorating, and/or Preventing Bacterial Infection

In some aspects, the present invention is directed to a method of treating, ameliorating and/or preventing a bacterial infection, such as in a subject in need thereof.

In some embodiments, the method includes administering to the subject an effective amount of a compound having the structure of Formula I:

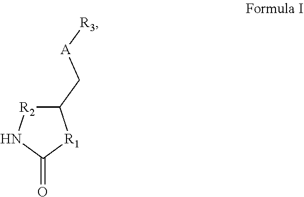

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

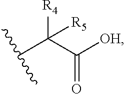

and
R₄ and R₅ are each independently H, halogen, C₁-C₆ alkyl, C₁-C₆ alkoxy, or —OH;

In some embodiments, the method includes administering to the subject an effective amount of a compound

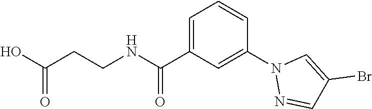 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

In some embodiments, in Formula I, A is

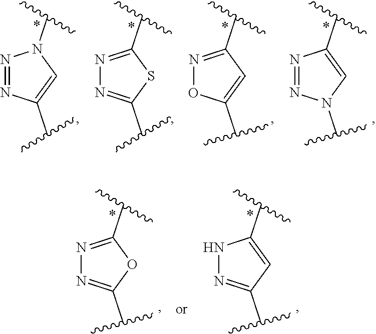

wherein * is the bond to R₃ and wherein the CH in the five-membered heterocyclyl group of A (if present) is independently optionally substituted with at least one of C₁-C₆ alkyl, C₁-C₆ alkoxy, and halogen.

In some embodiments, the compound of Formula I is at least one selected from the group consisting of:

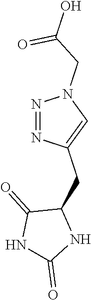 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

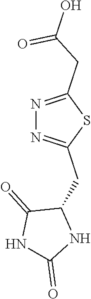 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

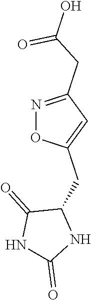 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

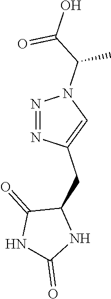 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

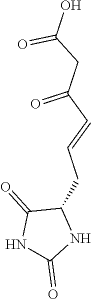 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

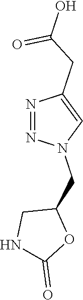 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

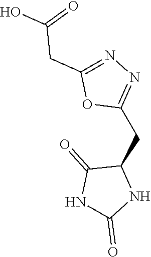 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

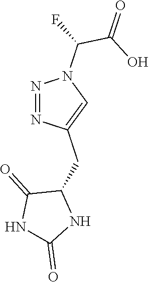 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

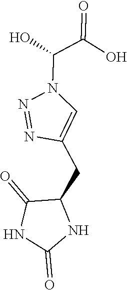 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

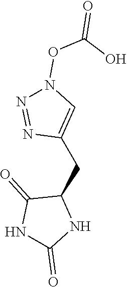 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

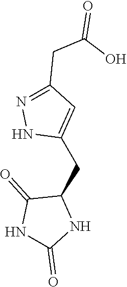 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

The compounds above, as well as the synthesis thereof, are described in WO 2023/049796 A1. The entirety of the reference is hereby incorporated herein by reference.

In some embodiments, the antibiotic resistance is derived from and/or involves formation of a biofilm or development of a stringent response by the bacterium.

In some embodiments, the bacterial infection is caused by an infection of a gram-positive bacterium or a gram-negative bacterium.

In some embodiments, the bacterial infection is caused by an infection of at least one of a B. burgdorferi bacterium, an E. coli bacterium, an H. influenzae bacterium, an N. gonorrhoeae bacterium, a P. aeruginosa bacterium, an S. epidermidis bacterium, an S. pneumoniae bacterium, and an S. aureus bacterium.

In some embodiments, the administration of the compound inhibits or reverses biofilm formation, inhibits a stringent response, inhibits toxin production, inhibits a hemolytic effect, reduces ability to protect against an oxidative stress, or reduces resistance to an antibiotic in the bacterium causing the infection.

In some embodiments, the bacterial infection causes a skin lesion, and the compound treats, ameliorates and/or prevents the skin lesion in the subject.

In some embodiments, the method further includes administering to the subject an antibiotic effective for treating, ameliorating and/or preventing the bacterial infection.

Non-limiting examples of antibiotics include the penicillins (such as amoxicillin, ampicillin, dicloxacillin, oxacillin, penicillin, piperacillin, and the like); the tetracyclines (such as demeclocycline, doxycycline, eravacycline, minocycline, omadacycline, sarecycline, tetracycline, and the like); the cephalosporins (such as cefaclor, cefadroxil, cefdinir, cephalexin, cefprozil, cefdinir, cefepime, cefiderocol, cefotaxime, cefotetan, ceftaroline, ceftazidime, ceftriaxone, cefuroxime, and the like); the fluoroquinolones (such as ciprofloxacin, delafloxacin, levofloxacin, moxifloxacin, gemifloxacin, and the like); the lincomycins (such as clindamycin, lincomycin, and the like); the macrolides (such as azithromycin, clarithromycin, erythromycin, and the like); the sulfonamides (such as sulfamethoxazole, sulfasalazine, and the like); the glycopeptide antibiotics (such as dalbavancin, oritavancin, telavancin, vancomycin, and the like); the aminoglycosides (such as gentamicin, tobramycin, amikacin, neomycin, and the like); the carbapenems (such as the imipenem, meropenem, ertapenem, and the like); polymyxins B and E; bacitracin; and the like.

In some embodiments, the subject is a mammal.

In some embodiments, the subject is a human.

### Method of Reducing or Eliminating Adverse Effect Caused by Bacteria

In some aspects, the present invention is directed to a method of reducing or eliminating an adverse effect caused by a bacterium in a subject.

In some embodiments, the method includes administering to the subject a compound, which is capable of inhibiting, reducing, and/or reversing a biofilm by the bacterium, reducing or eliminating production of a toxin by the bacterium, reducing or eliminating a hemolytic effect caused by the bacterium, and/or reducing or reversing a skin lesion caused by the bacterium.

In some embodiments, the compound inhibits RelA or other RelA/SpoT Homology (RSH) enzymes in the bacterium.

In some embodiments, the compound is the same as or similar to those described elsewhere herein, such as in the “Method of Treating, Ameliorating and/or Preventing Bacterial Infection” section.

In some embodiments, the method further includes administering to the subject an antibiotic effective for killing or inhibiting growth of the bacterium. In some embodiments, the antibiotic is the same as or similar to those described elsewhere herein, such as in the “Method of Treating, Ameliorating and/or Preventing Bacterial Infection” section.

In some embodiments, the subject is a mammal. In some embodiments, the subject is a human.

Method of Reducing Transformation, Horizontal Gene Transfer, and/or Acquisition of Antibiotic Resistance in Bacterium

In some aspects, the present invention is directed to a method of reducing or eliminating transformation, horizontal gene transfer (HGT) and/or acquisition of antibiotic resistance in a bacterium.

In some embodiments, the method comprises contacting the bacterium with the compounds herein.

### Compositions and Kits

In some aspects, the present study is directed to a composition or a kit.

In some embodiments, the composition or kit is for carrying out the methods disclosed herein.

In some embodiments, the composition of the kit includes the compound herein, and an antibiotic (the compounds and the antibiotics are the same as or similar to those as described in the “Method of Treating, Ameliorating and/or Preventing Bacterial Infection” section).

In some embodiments, the composition herein further includes a pharmaceutically acceptable carrier.

In some embodiments, the kit herein further includes a manual instructing that the compound and the antibiotic are to be administered to a subject infected with a bacterium, optionally an antibiotic-resistant bacterium, optionally a bacterium resistant to the antibiotic by forming a biofilm or via a stringent response.

### Pharmaceutical Compositions and Formulations

The instant specification provides pharmaceutical compositions comprising at least one compound of the instant specification or a salt or solvate thereof, which are useful to practice methods of the instant specification. Such a pharmaceutical composition may consist of at least one compound of the instant specification or a salt or solvate thereof, in a form suitable for administration to a subject, or the pharmaceutical composition may comprise at least one compound of the instant specification or a salt or solvate thereof, and one or more pharmaceutically acceptable carriers, one or more additional ingredients, or any combinations of these. At least one compound of the instant specification may be present in the pharmaceutical composition in the form of a physiologically acceptable salt, such as in combination with a physiologically acceptable cation or anion, as is well known in the art.

In some embodiments, the pharmaceutical compositions useful for practicing the method of the instant specification may be administered to deliver a dose of between 1 ng/kg/day and 100 mg/kg/day. In other embodiments, the pharmaceutical compositions useful for practicing the instant specification may be administered to deliver a dose of between 1 ng/kg/day and 1,000 mg/kg/day.

The relative amounts of the active ingredient, the pharmaceutically acceptable carrier, and any additional ingredients in a pharmaceutical composition of the instant specification will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the instant specification may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the instant specification may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In some embodiments, the compositions of the instant specification are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the instant specification is contemplated include, but are not limited to, humans and other primates, mammals including commercially relevant mammals such as cattle, pigs, horses, sheep, cats, and dogs.

In some embodiments, the compositions of the instant specification are formulated using one or more pharmaceutically acceptable excipients or carriers. In some embodiments, the pharmaceutical compositions of the instant specification comprise a therapeutically effective amount of at least one compound of the instant specification and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the instant specification may comprise a preservative from about 0.005% to 2.0% by total weight of the composition. The preservative is used to prevent spoilage in the case of exposure to contaminants in the environment. Examples of preservatives useful in accordance with the instant specification include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose.

Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the instant specification may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the instant specification may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the instant specification may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosage/Formulations

The regimen of administration may affect what constitutes an effective amount. The therapeutic formulations may be administered to the subject either prior to or after the onset of a disease or disorder that is affected by, associated with, or would benefit from antibacterial treatment. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat a disease or disorder that is affected by, associated with, or would benefit from antibacterial treatment in the patient. An effective amount of the therapeutic compound necessary to achieve a therapeutic effect may vary according to factors such as the state of the disease or disorder in the patient; the age, sex, and weight of the patient; and the ability of the therapeutic compound to treat a bacterial-based disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In various embodiments, the dose of a compound described herein is from about 1 mg and about 2,500 mg. In various embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in various embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, prevent, or reduce one or more symptoms in a patient of a disease or disorder that is affected by, associated with, or would benefit from antibacterial treatment.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any one of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% α-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

### Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of a disease or disorder that is affected by, associated with, or would benefit from antibacterial treatment in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

For the past eighty-plus years, beginning with the widespread introduction of penicillin by the US military in World War II, essentially all antibiotics developed have been designed to kill rapidly growing and dividing planktonic, single-celled bacteria. While this strategy has proven largely successful for the treatment of acute infections in the past, the widespread use of antibiotics has also driven the development of genetic antibiotic resistance mechanisms in bacteria.

A recent analysis by researchers from around the world (Murray et al., Lancet. 2022 Feb. 12; 399(10325):629-655) estimates that, in the year 2019, there were 4.95 million deaths associated with antimicrobial resistance (AMR) worldwide, of which 1.27 million deaths were directly attributable to AMR. To put things into perspective, the death figure reported in the article is greater than for any other non-COVID-related infectious disease deaths—including AIDS and malaria combined during this particular time period.

The rise of antimicrobial resistance and the lack of a viable counter-measurements further threaten the effectiveness of human medicine in areas such as joint replacements, Caesarean sections, organ transplants, chemotherapy, dialysis and more.

Beginning in the 1990s it was realized that most chronic bacterial infections had a multicellular biofilm component, which confers the bacteria with significant resistance to antibiotic treatments. Subsequently it was determined that this recalcitrance to standard antimicrobial therapies was due to metabolic quiescence; which allows bacteria in a biofilm to reach antibiotic resistance of more than a thousand-fold that of their planktonic counterparts.

It was discovered that bacterial persistence in the face of antibiotics comes from the stringent response. The stringent response converts bacterial metabolism from growth and replication to one survival. This is accomplished, in part, by the upregulation of metabolic pathways that protect against oxidative stress, which is the also the general mechanism by which virtually all antibiotics kill bacteria.

The roles of the stringent response in biofilm resistance to antibiotics is confirmed by knocking-out relA, a gene responsible for the stringent response, in Pseudomonas aeruginosa. After relA knock, P. aeruginosa would still be able to form robust biofilms but could be killed with antibiotics in a manner similar to planktonic bacteria.

In the present study, a group of organic compounds that inhibit relA protein were developed.

### Example 2

The ribosomally-associated enzyme RelA, upon sensing starvation, triggers the bacterial stringent response through production of the alarmone (p)ppGpp (also termed “magic spot”) which results in a shift in metabolism from growth and division to survival—characterized, in part, by upregulation of stress response mechanisms providing for resistance to reactive oxygen species (ROS). This provides for both the bacterial biofilm recalcitrance to antibiotics and bacterial survival in the phagolysosome of the cellular component of innate immunity.

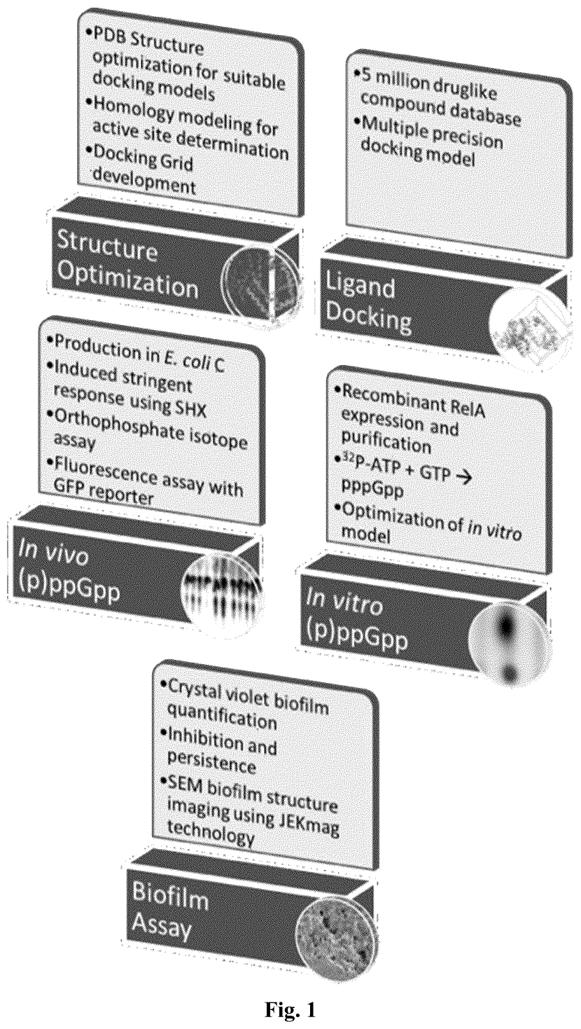

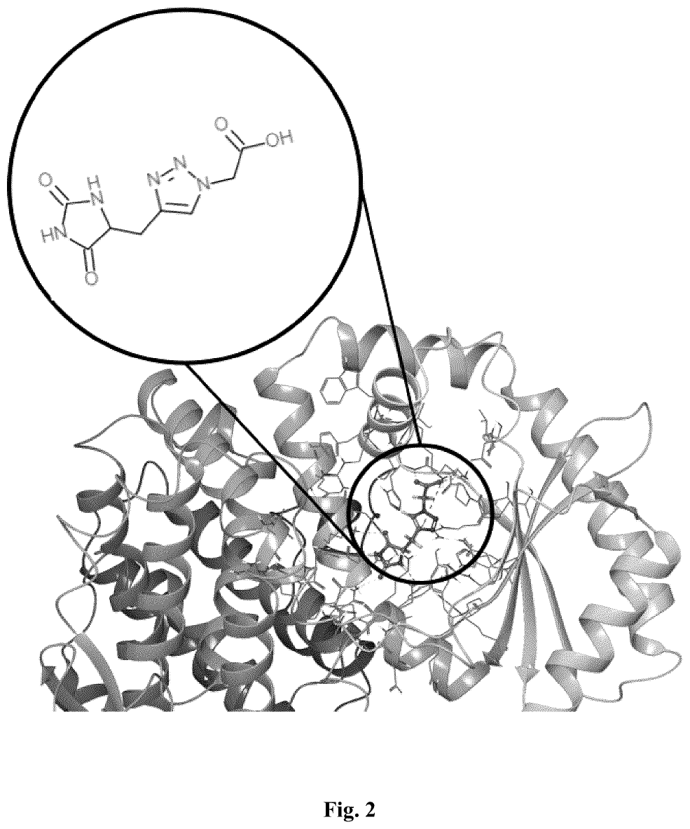

The present study developed a highly-effective hybrid computational-laboratory pipeline for identifying and characterizing small molecule inhibitors of the bacterial RelA enzyme and its homologs to serve as antimicrobial therapeutics (FIG. 1). More than 5×10⁶ druggable compounds were modeled, and anti-RelA compounds (FIG. 2) were designed as co-drugs for the re-potentiation of antibiotics against biofilms and bacterial survival in the phagolysosome.

### Example 3: HEJ14 Inhibits Transformation and Growth in Haemophilus influenzae

To study the effects of HEJ14, including the risk of resistance mutations arising, the present study constructed single gene knockouts of RelA, SpoT, and MsfI (a gene that provides for survival in phagolysosomes) as well as a double RelA/SpoT knockout in the Gram-negative bacteria Haemophilus influenzae using an overlap PCR mutagenesis strategy.

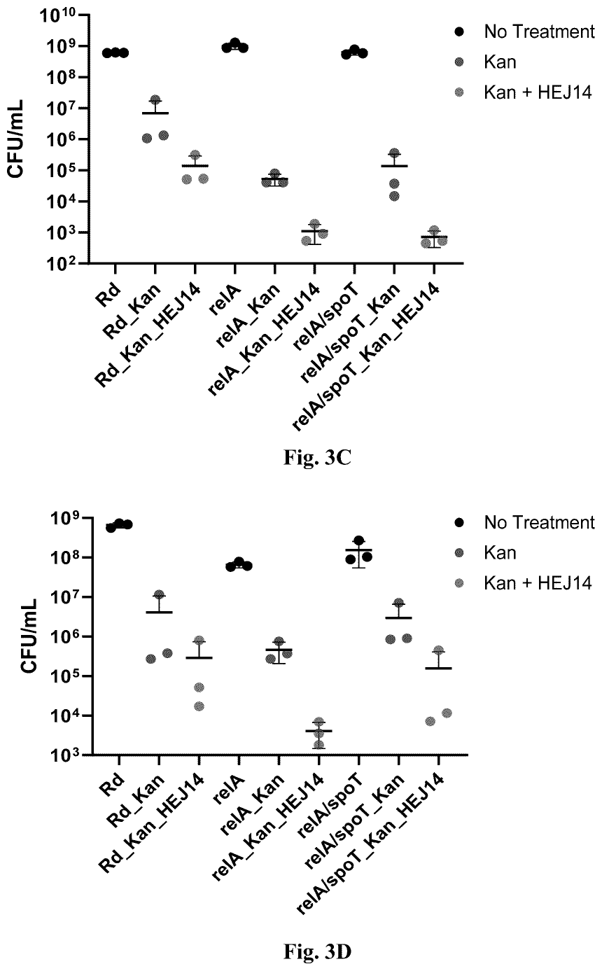

Referring to FIGS. 4A and 4B, both the ΔRelA and the ΔRelA/ΔSpot strains display a highly significant defect in transformational frequency, a major mechanism for acquisition of AMR genes. Considering that competence is induced in H. influenzae via a shift to a starvation media, it is reasoned that HEJ14 treatment would also reduce the acquisition of antimicrobial resistance in bacteria, as the inhibition of RelA would reduce horizontal gene transfer.

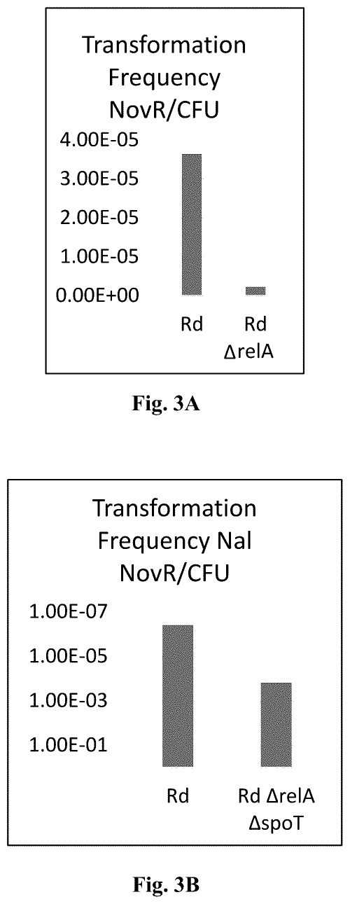

The present study then evaluated the susceptibility of both planktonic (FIG. 3C) and biofilm cells (FIG. 3D) of WT, ΔRelA and the ΔRelA/ΔSpot H. influenzae strains to kanamycin (kan) alone and in combination with HEJ14. In all cases there was a 2-3 log increase in killing by the combo treatment confirming our initial premise that RelA inhibition would potentiate standard antibiotics.

It is worth noting that HEJ14 was able to inhibit planktonic and biofilm H. influenzae growth even in ΔRelA strains (FIGS. 3C and 3D), indicating that HEJ14 acts through RelA-independent pathways, as well. [[Inventor: Is this correct?]]

### Example 4: Anti-Toxin Effect of RelA Inhibitors

Unexpectedly, the present study discovered that some of the hit compounds, as single agents, inhibited pyocyanin toxin production (FIG. 4) in the Gram-negative pathogen P. aeruginosa at higher specific activities (nanomolar) than required for RelA inhibition. Thus, the present study pursued a bipartite strategy of developing the compounds to treat biofilms, and to “convert” pathogens to commensals.

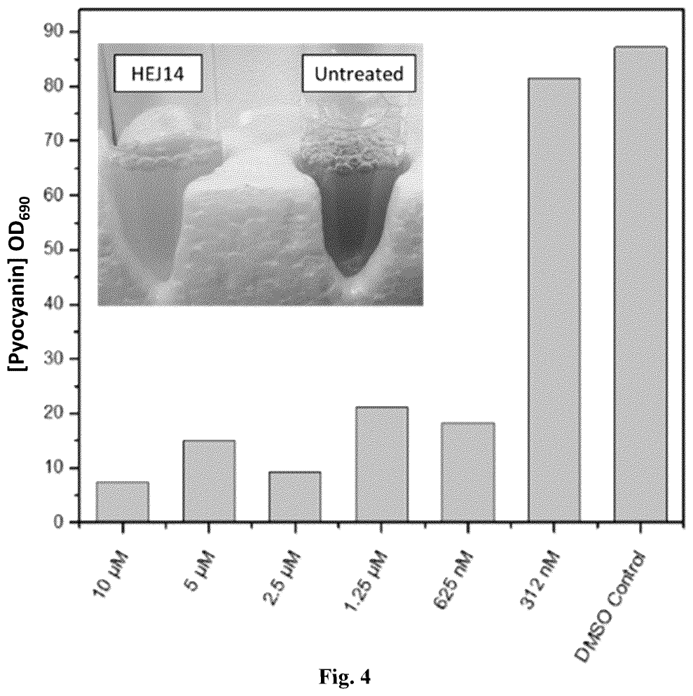

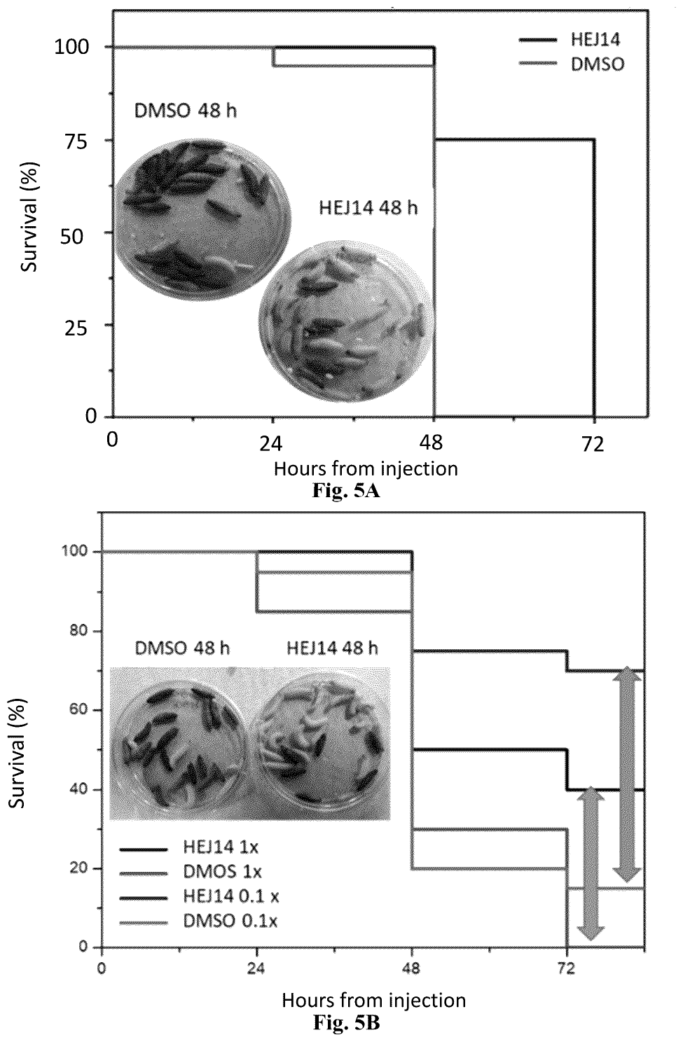

Importantly, compound HEJ14, significantly reduced P. aeruginosa virulence in the wax worm model when the larvae were injected with supernatant from P. aeruginosa cultures treated with HEJ14 during growth compared to untreated culture supernatants (FIG. 5A). Similarly, HEJ14 protected the larvae for 48 hours when live P. aeruginosa were injected simultaneously with a single bolus of HEJ14 (FIG. 5B). At 48 hours post-injection, 95% of the larvae treated with HEJ14 survived, whereas all the larvae treated with DMSO control died—demonstrating a highly significant efficacy.

### Example 5: HEJ14 Decreases Virulence of Staphylococcus aureus in a Vertebrate Pre-Clinical Model

The present study further discovered that the bacterial anti-virulence effects of HEJ14 works on the Gram-positive pathogens, as well. To determine if HEJ14 has an effect on the Gram-positive bacteria Staphylococcus aureus, the present study performed in vitro growth kinetics assays testing various HEJ14 concentrations and measuring OD₆₀₀ over time.

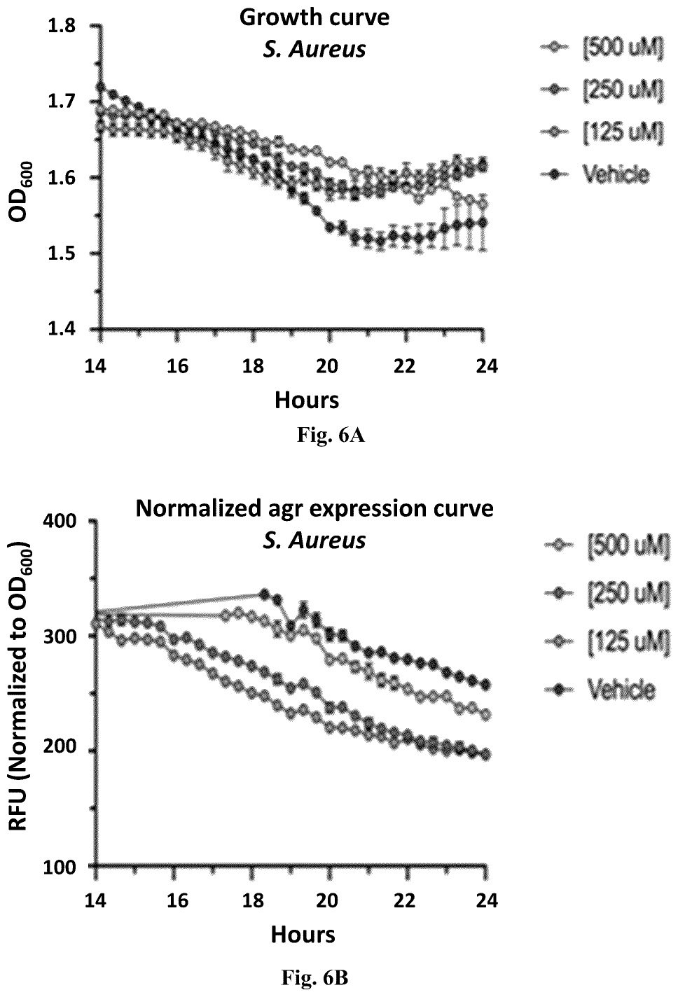

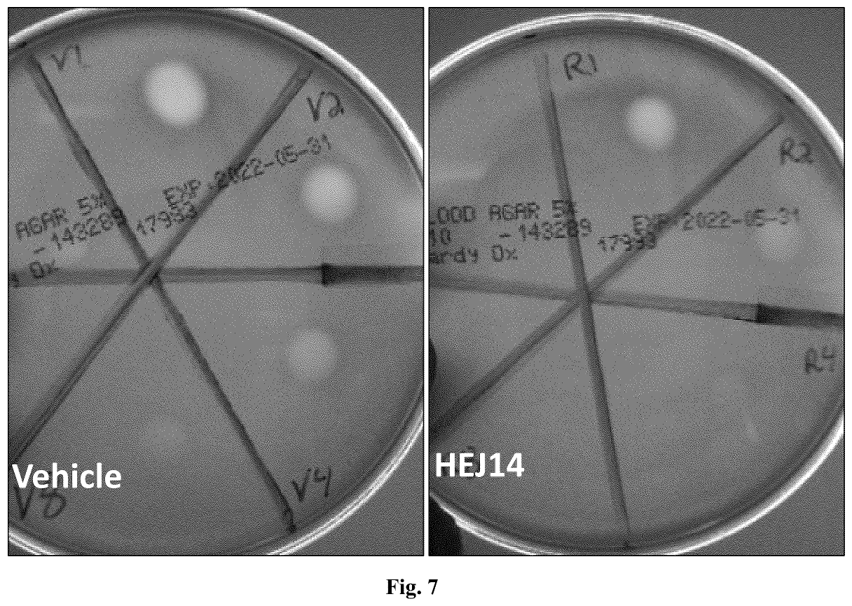

The present study observed that HEJ14 enhanced bacterial growth during the stationary phase (FIG. 6A), indicative of an inhibited stringent response. Since the stringent response in S. aureus induces PSMα production and agr activity is essential for psm expression, the present study used an S. aureus agr reporter strain to elucidate the effect of RelA inhibition on agr expression. The present study discovered that HEJ14 caused a dose-dependent decrease in agr expression during stationary phase (FIG. 6B).

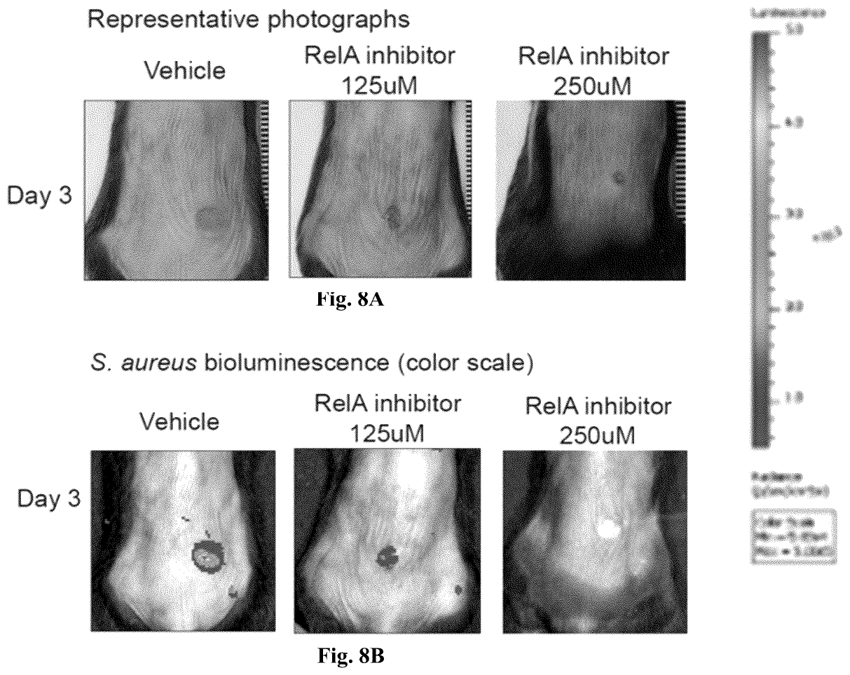

Furthermore, supernatants from HEJ14-treated S. aureus cultures have reduced hemolytic effect on blood agar plates as compared to supernatants from vehicle treated S. aureus cultures (FIG. 7).

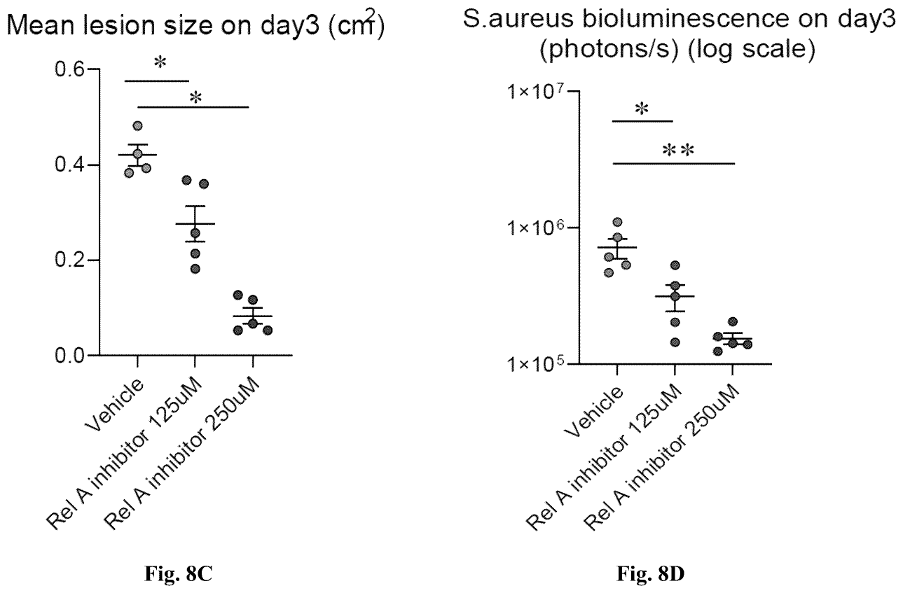

To investigate the efficacy of a RelA inhibitor against in vivo bacterial infections, the present study employed an established mouse model of S. aureus intradermal infection with the community-associated methicillin-resistant S. aureus infections (CA-MRSA) bioluminescent strain USA300 LAC::lux (Marchitto et al., Proc Natl Acad Sci USA 2019; 116:10917-26; Alphonse et al., Sci Transl Med 2021; 13.; Wang et al., Nat Med 2007; 13:1510-4.; Dong et al., Immunity 2022; 55:1645-62.e7). The present study found that mice treated with a single dose of 125 μM HEJ14 or 250 μM HEJ14 at the time of infection had significantly decreased bioluminescence (as a measure of in vivo bacterial burden) and lesion size compared to vehicle-treated mice on day 3, in a dose-dependent manner (FIGS. 8A-8D).

Taken together, these data demonstrate that the RelA inhibitors described herein restrain S. aureus agr expression and toxin production, and show efficacy as an anti-microbial agent against in vivo methicillin-resistant S. aureus (MRSA) infections.

### Example 6: Compound HEJ14 Eliminates Bacterial Aggregation and Reduces Biofilm Formation in Escherichia coli C

E. coli C, when under certain stress conditions, aggregates together and forms highly robust biofilms from a planktonic culture. It was hypothesized that inhibition of RelA would limit aggregation of E. coli C.

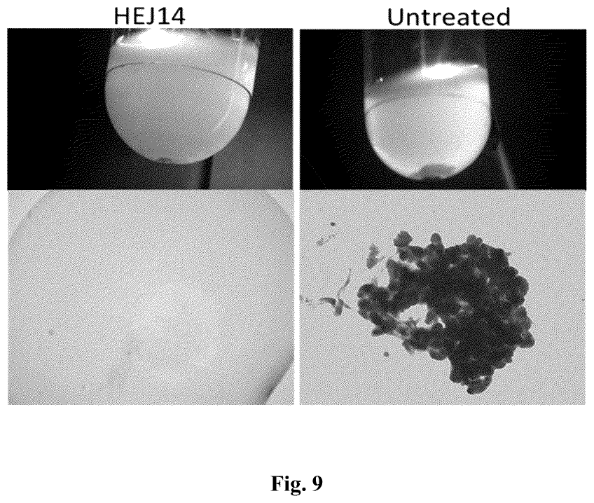

The present study found that HEJ14 reduced the amount of E. coli C aggregation under high salt, low temperature and amino acid starvation (FIG. 9).

### Example 7: Enhancement of Antibiotic-Killing by RelA Inhibitors

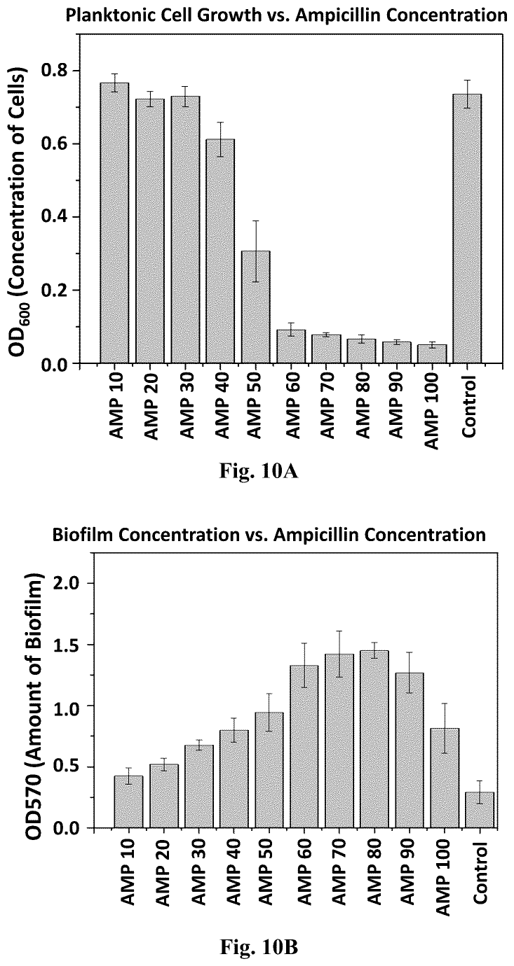

Biofilm formations are markedly enhanced in the presence of antibiotics at concentrations far exceeding those needed to eliminate planktonic growth of the bacteria (FIGS. 10A-10B).

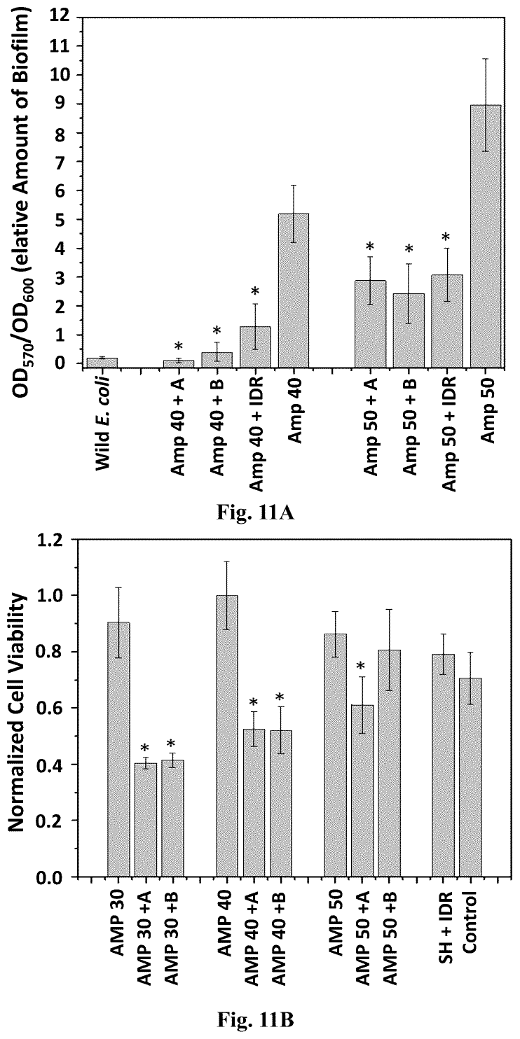

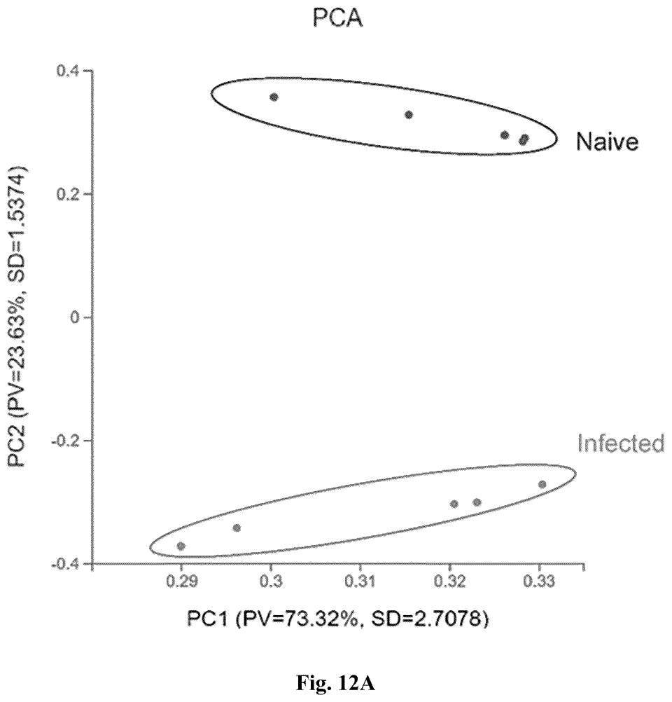

The present study thus evaluated the effects of antibiotic in the presence or absence of some RelA inhibitors (S1-G1A [A]& S1-G1B [B]) described herein (FIGS. 11A-11B).

Supplementing antibiotics with the relA inhibitors significantly reduced biofilm mass and bacterial cell viability counts compared to both antibiotics alone and relA inhibitor (IDR) alone.

### Example 7: RNAseq Studies of the Murine Model of Dermal Infection

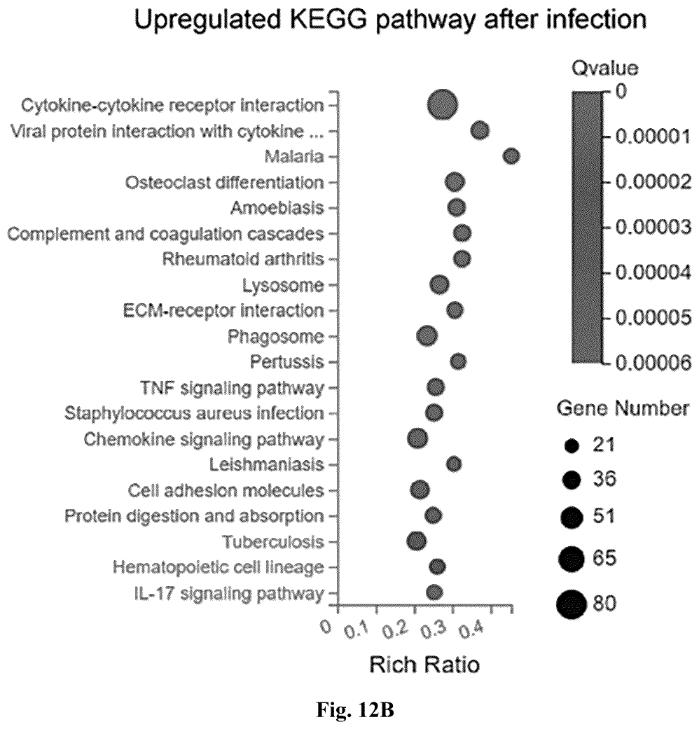

Based on HEJ14's efficacy in the murine model of Sa infection, the present study sought to characterize gene expression patterns of host and microbe in our treatment models. The present study generated RNAseq data from the untreated dermal Sa model of infection (FIGS. 12A-12B).

### Enumerated Embodiments

In some aspects, the present invention is directed to the following non-limiting embodiments:

Embodiment 1: A method of treating, ameliorating, and/or preventing a bacterial infection in a subject in need thereof, the method comprising administering to the subject an effective amount of at least one compound selected from:
(a) a compound of Formula I:

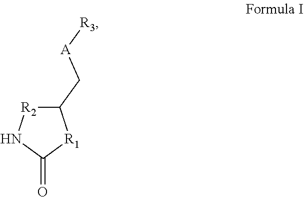

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

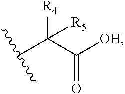

and
R₄ and R₅ are each independently H, halogen, C₁-C₆ alkyl, C₁-C₆ alkoxy, or —OH;
(b)

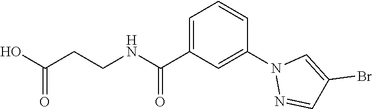 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

Embodiment 2: The method of Embodiment 1, wherein in Formula I, A is

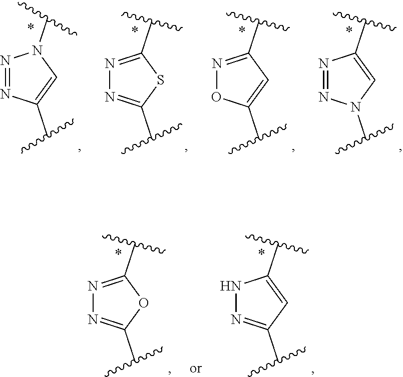 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

Embodiment 3: The method of any one of Embodiments 1-2, wherein the compound of Formula I is at least one selected from the group consisting of:

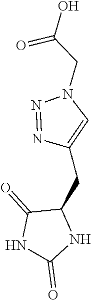 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

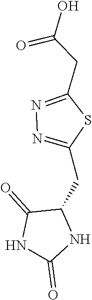 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C4-G2A);

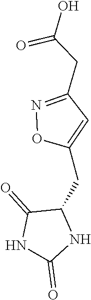 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

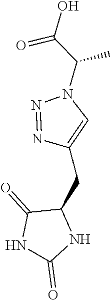 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

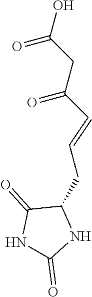 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

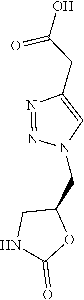 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

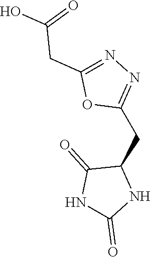 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

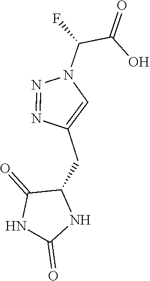 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

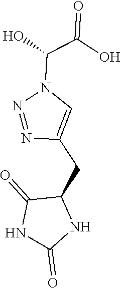 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

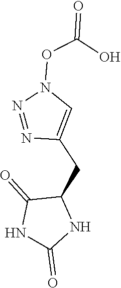 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

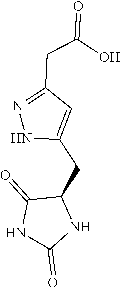 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

Embodiment 4: The method of any one of Embodiments 1-3, wherein the bacterial infection is caused by an antibiotic resistant bacterium.

Embodiment 5: The method of Embodiment 4, wherein the antibiotic resistance of the bacterium is derived from and/or involves formation of a biofilm or development of a stringent response by the bacterium.

Embodiment 6: The method of any one of Embodiments 1-5, wherein the bacterium is a gram-positive bacterium or a gram-negative bacterium.

Embodiment 7: The method of any one of Embodiments 1-6, wherein the bacterium is at least one of B. burgdorferi, E. coli, H. influenzae, N. gonorrhoeae, P. aeruginosa, S. epidermidis, S. pneumoniae, and S. aureus.

Embodiment 8: The method of any one of Embodiments 1-7, wherein the administration of the compound inhibits or reverses biofilm formation, inhibits a stringent response, inhibits production of a toxin, inhibits a hemolytic effect, reduces ability to protect against an oxidative stress, reduces antibiotic resistance, reduces the acquisition of antibiotic resistance, reduces horizontal gene transfer, and/or reduce transformation in the bacterium causing the infection.

Embodiment 9: The method of any one of Embodiments 1-8, wherein the bacterial infection causes a skin lesion, and wherein the compound treats, ameliorates and/or prevents the skin lesion in the subject.

Embodiment 10: The method of any one of Embodiments 1-9, further comprises administering to the subject an antibiotic effective for treating, ameliorating and/or preventing the bacterial infection.

Embodiment 11: The method of any one of Embodiments 1-10, wherein the subject is a mammal, optionally a human.

Embodiment 12: A method of reducing or eliminating an adverse effect caused by a bacterium in a subject, wherein the adverse effect is at least one selected from the group consisting of biofilm formation, toxin production, a hemolytic effect, and a skin lesion formation,
the method comprising administering to the subject an effective amount of at least one compound selected from:
(a) a compound of Formula I:

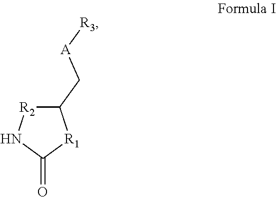

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

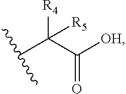

and
R₄ and R₅ are each independently H, halogen, C₁-C₆ alkyl, C₁-C₆ alkoxy, or —OH;
(b)

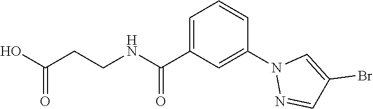 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

Embodiment 13: The method of Embodiment 12, wherein in Formula I, A is

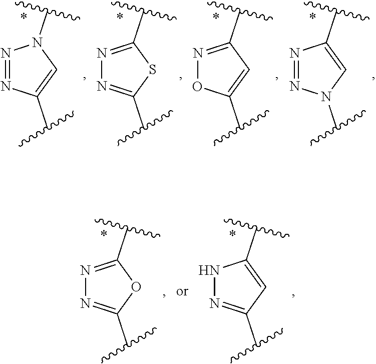 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

Embodiment 14: The method of any one of Embodiments 12-13, wherein the compound of Formula I is at least one selected from the group consisting of:

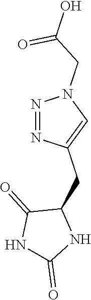 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

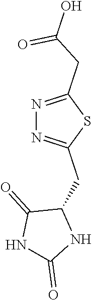 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

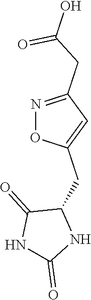 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

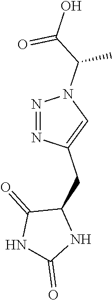 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

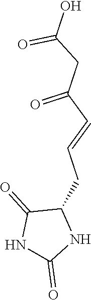 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

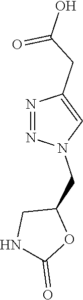 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C4-G2E);

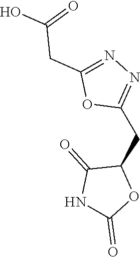 [SMILES: O=C(O)Cc1nnc(C[C@H]2OC(=O)NC2=O)o1]

(R)-2-(5-((2,5-dioxoimidazolidin-4-yl)methyl)-1,3,4-oxadiazol-2-yl)acetic acid (C14-G2F);

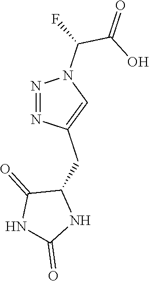 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

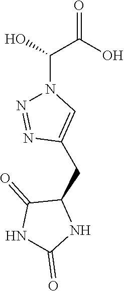 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

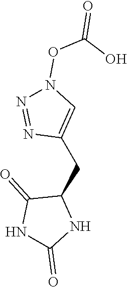 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

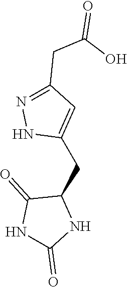 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

Embodiment 15: The method of any one of Embodiments 12-14, wherein the bacterium is an antibiotic-resistant bacterium.

Embodiment 16: The method of Embodiment 15, wherein the antibiotic resistance of the bacterium is derived from and/or involves formation of a biofilm or development of a stringent response by the bacterium.

Embodiment 17: The method of any one of Embodiments 12-16, wherein the bacterium is a gram-positive bacterium or a gram-negative bacterium.

Embodiment 18: The method of any one of Embodiments 12-17, wherein the bacterium is at least one of B. burgdorferi, E. coli, H. influenzae, N. gonorrhoeae, P. aeruginosa, S. epidermidis, S. pneumoniae, and S. aureus.

Embodiment 19: The method of any one of Embodiments 12-18, further comprises administering to the subject an antibiotic effective for killing or inhibiting growth of the bacterium.

Embodiment 20: The method of any one of Embodiments 12-19, wherein the subject is a mammal, optionally a human.

Embodiment 21: A composition, comprising:

at least one compound selected from:
(a) a compound of Formula I:

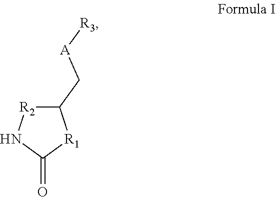

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

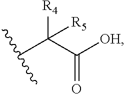

and
R₄ and R₅ are each independently H, halogen, C₁-C₆ alkyl, C₁-C₆ alkoxy, or —OH;
(b)

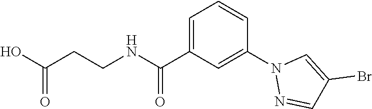 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof;
   and
   an antibiotic.

Embodiment 22: The composition of Embodiment 21, wherein in Formula I, A is

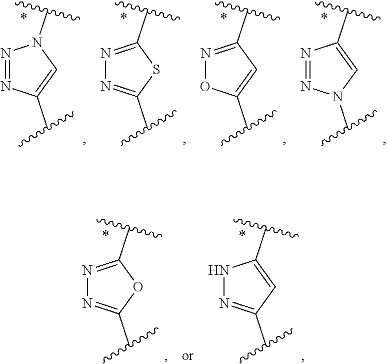 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen

Embodiment 23: The composition of any one of Embodiments 21-22, wherein the compound of Formula I is at least one selected from the group consisting of:

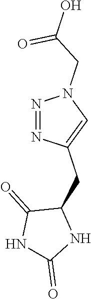 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

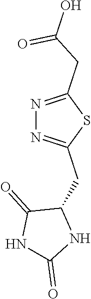 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

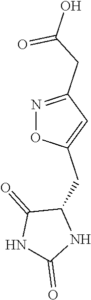 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

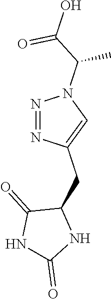 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

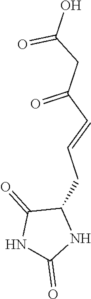 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

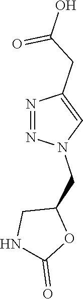 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

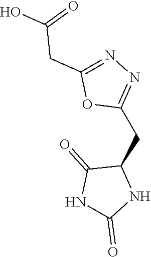 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

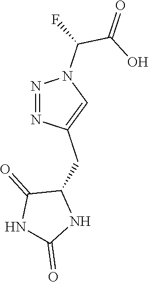 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

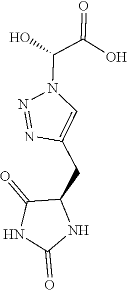 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

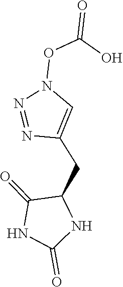 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

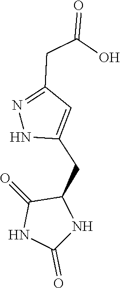 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

Embodiment 24: The composition of any one of Embodiments 21-23, wherein the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

Embodiment 25: The composition of any one of Embodiments 21-24, further comprises a pharmaceutically acceptable carrier.

Embodiment 26: A kit, comprising:

at least one compound selected from:
(a) a compound of Formula I:

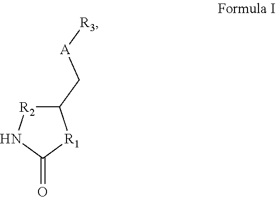

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

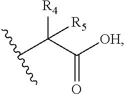

and
R₄ and R₅ are each independently H, halogen, C₁-C₆ alkyl, C₁-C₆ alkoxy, or —OH;
(b)

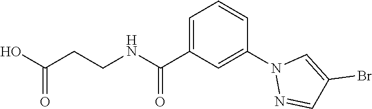 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof;
   and
   an antibiotic.

Embodiment 27: The kit of Embodiment 26, wherein in Formula I, A is

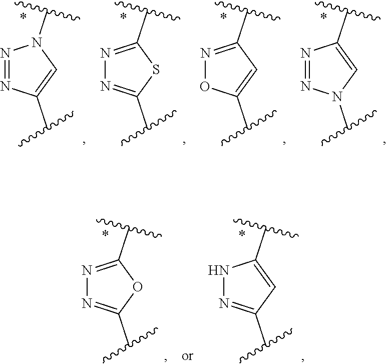 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen

Embodiment 28: The kit of any one of Embodiments 26-27, wherein the compound of Formula I is at least one selected from the group consisting of:

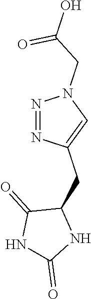 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

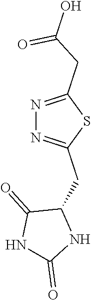 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

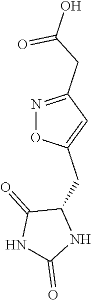 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

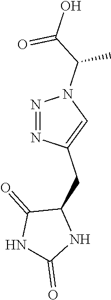 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

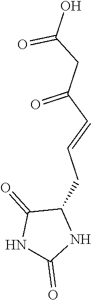 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

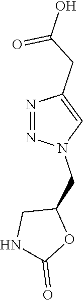 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

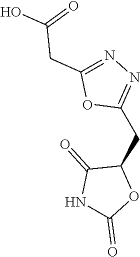 [SMILES: O=C(O)Cc1nnc(C[C@H]2OC(=O)NC2=O)o1]

(R)-2-(5-((2,5-dioxoimidazolidin-4-yl)methyl)-1,3,4-oxadiazol-2-yl)acetic acid (C14-G2F);

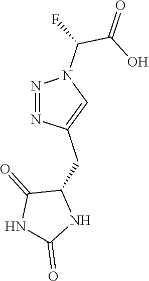 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

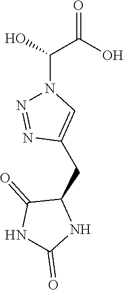 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

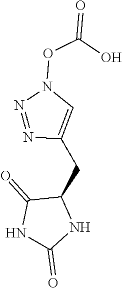 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

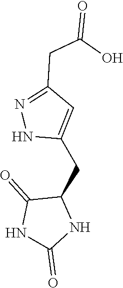 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

Embodiment 29: The kit of any one of Embodiments 26-28, wherein the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

Embodiment 30: The kit of any one of Embodiments 26-29, further comprises a manual instructing that the compound and the antibiotic are to be administered to a subject infected with a bacterium, optionally an antibiotic-resistant bacterium.

Embodiment 31: A method of reducing the acquisition of antibody resistance in a bacterium, the method comprising contacting the bacterium with at least one compound selected from:
(a) a compound of Formula I:

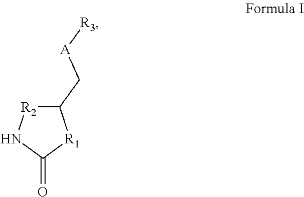

or a salt, solvate, tautomer, N-oxide, geometric isomer, stereoisomer thereof, and/or mixtures thereof, wherein:
   R₁ is —NH— or —O—,
   R₂ is —CH₂— or —C(O)—,
   A is a five member aromatic heterocyclic ring or —CH═CH—COO—*, wherein * is the bond to R₃.
   R₃ is —O—C(O)OH or

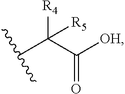

and
R₄ and R₅ are each independently H, halogen, C₁-C₆ alkyl, C₁-C₆ alkoxy, or —OH;
(b)

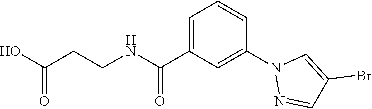 [SMILES: O=C(O)CCNC(=O)c1cccc(-n2cc(Br)cn2)c1]

3-(3-(4-bromo-1H-pyrazol-1-yl)benzamido)propanoic acid (C22), or a salt, solvate, tautomer, N-oxide, geometric isomer, and/or mixtures thereof.

Embodiment 32: The method of Embodiment 31, wherein in Formula I, A is

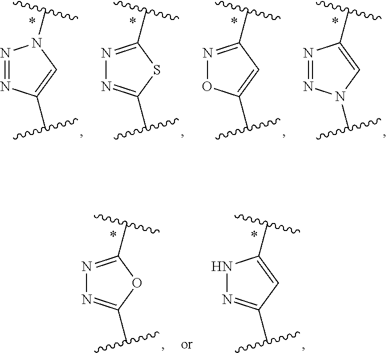 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]

wherein * is the bond to R₃ and wherein the CH in the five-membered heterocyclyl group of A (if present) is independently optionally substituted with at least one of C₁-C₆ alkyl, C₁-C₆ alkoxy, and halogen.

Embodiment 33: The method of any one of Embodiments 31-32, wherein the compound of Formula I is at least one selected from the group consisting of:

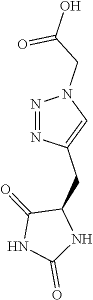 [SMILES: O=C(O)Cn1cc(C[C@H]2NC(=O)NC2=O)nn1]

(R)-2-(4-((2,5-dioxoimidazolidin-4-yl)methyl)-1H-1,2,3-triazol-1-yl)acetic acid (C14 or HEJ14);

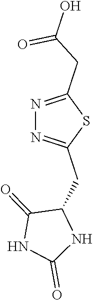 [SMILES: O=C(O)Cc1nnc(C[C@@H]2NC(=O)NC2=O)s1]

(S)-2-(5-((2,5-dioxoimidazolidin-4-yl)methyl)-1,3,4-thiadiazol-2-yl)acetic acid (C14-G2A);

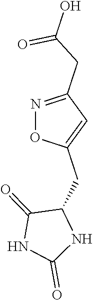 [SMILES: O=C(O)Cc1cc(C[C@@H]2NC(=O)NC2=O)on1]

(S)-2-(5-((2,5-dioxoimidazolidin-4-yl)methyl)isoxazol-3-yl)acetic acid (C14-G2B);

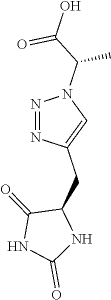 [SMILES: C[C@@H](C(=O)O)n1cc(C[C@H]2NC(=O)NC2=O)nn1]

(S)-2-(4-(((R)-2,5-dioxoimidazolidin-4-yl)methyl)-1H-1,2,3-triazol-1-yl)propanoic acid (C14-G2C);

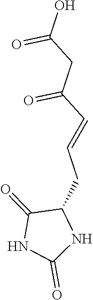 [SMILES: O=C(O)CC(=O)/C=C/C[C@@H]1NC(=O)NC1=O]

(S,E)-6-(2,5-dioxoimidazolidin-4-yl)-3-oxohex-4-enoic acid (C14-G2D);

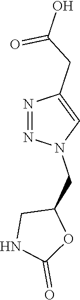 [SMILES: O=C(O)Cc1cn(C[C@@H]2CNC(=O)O2)nn1]

(S)-2-(1-((2-oxooxazolidin-5-yl)methyl)-1H-1,2,3-triazol-4-yl)acetic acid (C14-G2E);

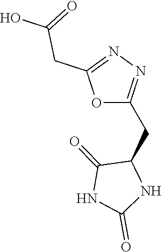 [SMILES: O=C(O)Cc1nnc(C[C@H]2NC(=O)NC2=O)o1]

(R)-2-(5-((2,5-dioxoimidazolidin-4-yl)methyl)-1,3,4-oxadiazol-2-yl)acetic acid (C14-G2F);

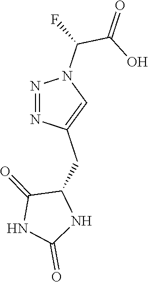 [SMILES: O=C1NC(=O)[C@H](Cc2cn([C@H](F)C(=O)O)nn2)N1]

(R)-2-(4-(((S)-2,5-dioxoimidazolidin-4-yl)methyl)-1H-1,2,3-triazol-1-yl)-2-fluoroacetic acid (C14-G2G);

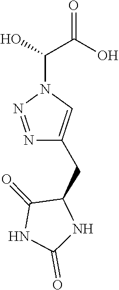 [SMILES: O=C1NC(=O)[C@@H](Cc2cn([C@H](O)C(=O)O)nn2)N1]

(R)-2-(4-(((R)-2,5-dioxoimidazolidin-4-yl)methyl)-1H-1,2,3-triazol-1-yl)-2-hydroxyacetic acid (C14-G2H);

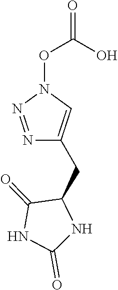 [SMILES: O=C1NC(=O)[C@@H](Cc2cn(OC(=O)O)nn2)N1]

(R)-4-((2,5-dioxoimidazolidin-4-yl)methyl)-1H-1,2,3-triazol-1-yl hydrogen carbonate (C14-G2I); and

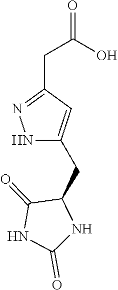 [SMILES: O=C(O)Cc1cc(C[C@H]2NC(=O)NC2=O)[nH]n1]

(R)-2-(5-((2,5-dioxoimidazolidin-4-yl)methyl)-1H-pyrazol-3-yl)acetic acid (C14-G2J).

Embodiment 34: The method of any one of Embodiments 31-33, further comprising contacting the bacterium with an antibiotic.

Embodiment 35: The method of Embodiment 34, wherein the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

Embodiment 36: The method of any one of Embodiments 31-35, wherein the bacterium is at least one selected from the group consisting of B. burgdorferi, E. coli, H. influenzae, N. gonorrhoeae, P. aeruginosa, S. epidermidis, S. pneumoniae, and S. aureus.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of treating, ameliorating, and/or preventing a bacterial infection in a subject in need thereof, the method comprising administering to the subject an effective amount of at least one compound selected from:
(a) a compound of Formula I: 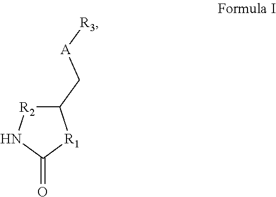
or a salt, solvate, tautomer, N-oxide, geometric isomer, stereoisomer thereof, and/or mixtures thereof, wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 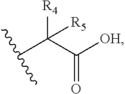
and
R₄ and R₅ are each independently H, halogen, C₁-C₆ alkyl, C₁-C₆ alkoxy, or —OH;
(b) 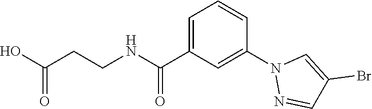 [SMILES: O=C(O)CCNC(=O)c1cccc(-n2cc(Br)cn2)c1]
3-(3-(4-bromo-1H-pyrazol-1-yl)benzamido)propanoic acid (C22), or a salt, solvate, tautomer, N-oxide, geometric isomer, and/or mixtures thereof.

2. The method of claim 1, wherein in Formula I, A is 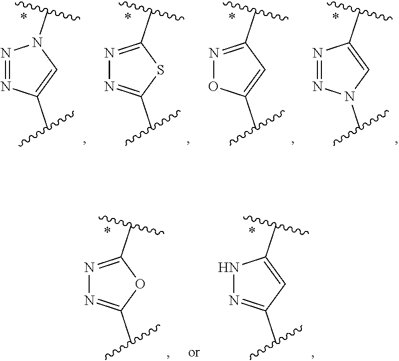 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]
wherein * is the bond to R₃ and wherein the CH in the five-membered heterocyclyl group of A (if present) is independently optionally substituted with at least one of C₁-C₆ alkyl, C₁-C₆ alkoxy, and halogen.

3. The method of claim 1, wherein the compound of Formula I is at least one selected from the group consisting of: 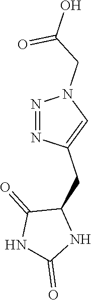 [SMILES: O=C(O)Cn1cc(C[C@H]2NC(=O)NC2=O)nn1]
(R)-2-(4-((2,5-dioxoimidazolidin-4-yl)methyl)-1H-1,2,3-triazol-1-yl)acetic acid (C14 or HEJ14); 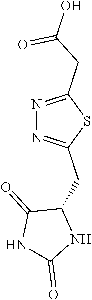 [SMILES: O=C(O)Cc1nnc(C[C@@H]2NC(=O)NC2=O)s1]
(S)-2-(5-((2,5-dioxoimidazolidin-4-yl)methyl)-1,3,4-thiadiazol-2-yl)acetic acid (C14-G2A); 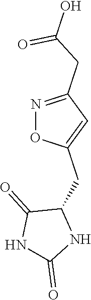 [SMILES: O=C(O)Cc1cc(C[C@@H]2NC(=O)NC2=O)on1]
(S)-2-(5-((2,5-dioxoimidazolidin-4-yl)methyl)isoxazol-3-yl)acetic acid (C14-G2B); 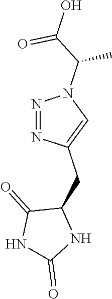 [SMILES: C[C@@H](C(=O)O)n1cc(C[C@H]2NC(=O)NC2=O)nn1]
(S)-2-(4-(((R)-2,5-dioxoimidazolidin-4-yl)methyl)-1H-1,2,3-triazol-1-yl)propanoic acid (C14-G2C); 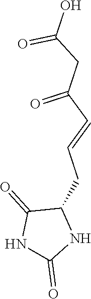 [SMILES: O=C(O)CC(=O)/C=C/C[C@@H]1NC(=O)NC1=O]
(S,E)-6-(2,5-dioxoimidazolidin-4-yl)-3-oxohex-4-enoic acid (C14-G2D); 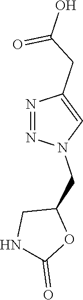 [SMILES: O=C(O)Cc1cn(C[C@@H]2CNC(=O)O2)nn1]
(S)-2-(1-((2-oxooxazolidin-5-yl)methyl)-1H-1,2,3-triazol-4-yl)acetic acid (C14-G2E); 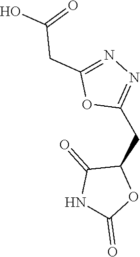 [SMILES: O=C(O)Cc1nnc(C[C@H]2OC(=O)NC2=O)o1]
(R)-2-(5-((2,5-dioxoimidazolidin-4-yl)methyl)-1,3,4-oxadiazol-2-yl)acetic acid (C14-G2F); 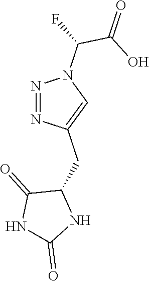 [SMILES: O=C1NC(=O)[C@H](Cc2cn([C@H](F)C(=O)O)nn2)N1]
(R)-2-(4-(((S)-2,5-dioxoimidazolidin-4-yl)methyl)-1H-1,2,3-triazol-1-yl)-2-fluoroacetic acid (C14-G2G); 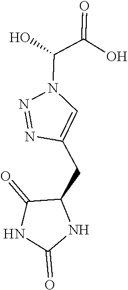 [SMILES: O=C1NC(=O)[C@@H](Cc2cn([C@H](O)C(=O)O)nn2)N1]
(R)-2-(4-(((R)-2,5-dioxoimidazolidin-4-yl)methyl)-1H-1,2,3-triazol-1-yl)-2-hydroxyacetic acid (C14-G2H); 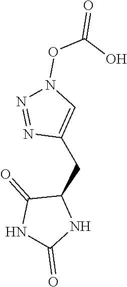 [SMILES: O=C1NC(=O)[C@@H](Cc2cn(OC(=O)O)nn2)N1]
(R)-4-((2,5-dioxoimidazolidin-4-yl)methyl)-1H-1,2,3-triazol-1-yl hydrogen carbonate (C14-G2I); and 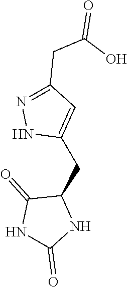 [SMILES: O=C(O)Cc1cc(C[C@H]2NC(=O)NC2=O)[nH]n1]
(R)-2-(5-((2,5-dioxoimidazolidin-4-yl)methyl)-1H-pyrazol-3-yl)acetic acid (C14-G2J).

4. The method of claim 1, wherein the bacterial infection is caused by an antibiotic resistant bacterium.

5. The method of claim 4, wherein the antibiotic resistance of the bacterium is derived from and/or involves formation of a biofilm or development of a stringent response by the bacterium.

6. The method of claim 1, wherein the bacterium is a gram-positive bacterium or a gram-negative bacterium.

7. The method of claim 1, wherein the bacterium is at least one of B. burgdorferi, E. coli, H. influenzae, N. gonorrhoeae, P. aeruginosa, S. epidermidis, S. pneumoniae, and S. aureus.

8. The method of claim 1, wherein the administration of the compound inhibits or reverses biofilm formation, inhibits a stringent response, inhibits production of a toxin, inhibits a hemolytic effect, reduces ability to protect against an oxidative stress, reduces antibiotic resistance, reduces the acquisition of antibiotic resistance, reduces horizontal gene transfer, and/or reduce transformation in the bacterium causing the infection.

9. The method of claim 1, wherein the bacterial infection causes a skin lesion, and wherein the compound treats, ameliorates and/or prevents the skin lesion in the subject.

10. The method of claim 1, further comprising administering to the subject an antibiotic effective for treating, ameliorating and/or preventing the bacterial infection.

11. The method of claim 1, wherein the subject is a mammal, optionally a human.

12. A method of reducing or eliminating an adverse effect caused by a bacterium in a subject, wherein the adverse effect is at least one selected from the group consisting of biofilm formation, toxin production, a hemolytic effect, and a skin lesion formation,
the method comprising administering to the subject an effective amount of at least one compound selected from:
(a) a compound of Formula I: 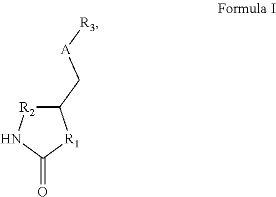
or a salt, solvate, tautomer, N-oxide, geometric isomer, stereoisomer thereof, and/or mixtures thereof, wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 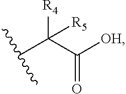
and
R₄ and R₅ are each independently H, halogen, C₁-C₆ alkyl, C₁-C₆ alkoxy, or —OH;
(b) 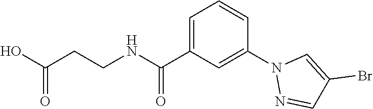 [SMILES: O=C(O)CCNC(=O)c1cccc(-n2cc(Br)cn2)c1]
3-(3-(4-bromo-1H-pyrazol-1-yl)benzamido)propanoic acid (C22), or a salt, solvate, tautomer, N-oxide, geometric isomer, and/or mixtures thereof.

13. The method of claim 12, wherein in Formula I, A is 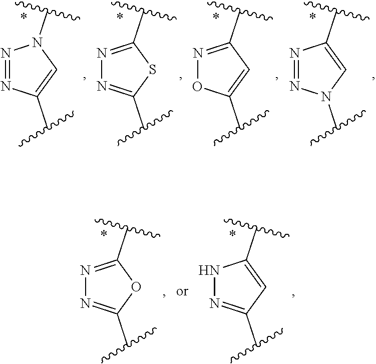 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]
wherein * is the bond to R₃ and wherein the CH in the five-membered heterocyclyl group of A (if present) is independently optionally substituted with at least one of C₁-C₆ alkyl, C₁-C₆ alkoxy, and halogen.

14. The method of claim 12, wherein the compound of Formula I is at least one selected from the group consisting of: 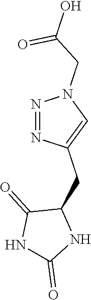 [SMILES: O=C(O)Cn1cc(C[C@H]2NC(=O)NC2=O)nn1]
(R)-2-(4-((2,5-dioxoimidazolidin-4-yl)methyl)-1H-1,2,3-triazol-1-yl)acetic acid (C14 or HEJ14); 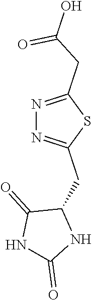 [SMILES: O=C(O)Cc1nnc(C[C@@H]2NC(=O)NC2=O)s1]
(S)-2-(5-((2,5-dioxoimidazolidin-4-yl)methyl)-1,3,4-thiadiazol-2-yl)acetic acid (C14-G2A); 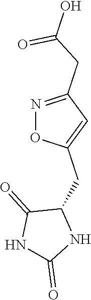 [SMILES: O=C(O)Cc1cc(C[C@@H]2NC(=O)NC2=O)on1]
(S)-2-(5-((2,5-dioxoimidazolidin-4-yl)methyl)isoxazol-3-yl)acetic acid (C14-G2B); 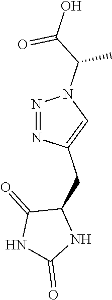 [SMILES: C[C@@H](C(=O)O)n1cc(C[C@H]2NC(=O)NC2=O)nn1]
(S)-2-(4-(((R)-2,5-dioxoimidazolidin-4-yl)methyl)-1H-1,2,3-triazol-1-yl)propanoic acid (C14-G2C); 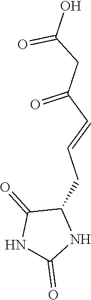 [SMILES: O=C(O)CC(=O)/C=C/C[C@@H]1NC(=O)NC1=O]
(S,E)-6-(2,5-dioxoimidazolidin-4-yl)-3-oxohex-4-enoic acid (C14-G2D); 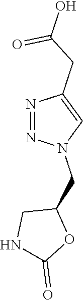 [SMILES: O=C(O)Cc1cn(C[C@@H]2CNC(=O)O2)nn1]
(S)-2-(1-((2-oxooxazolidin-5-yl)methyl)-1H-1,2,3-triazol-4-yl)acetic acid (C14-G2E); 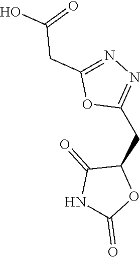 [SMILES: O=C(O)Cc1nnc(C[C@H]2OC(=O)NC2=O)o1]
(R)-2-(5-((2,5-dioxoimidazolidin-4-yl)methyl)-1,3,4-oxadiazol-2-yl)acetic acid (C14-G2F); 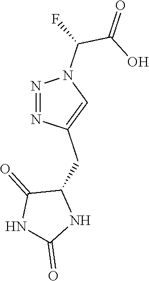 [SMILES: O=C1NC(=O)[C@H](Cc2cn([C@H](F)C(=O)O)nn2)N1]
(R)-2-(4-(((S)-2,5-dioxoimidazolidin-4-yl)methyl)-1H-1,2,3-triazol-1-yl)-2-fluoroacetic acid (C14-G2G); 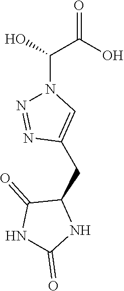 [SMILES: O=C1NC(=O)[C@@H](Cc2cn([C@H](O)C(=O)O)nn2)N1]
(R)-2-(4-(((R)-2,5-dioxoimidazolidin-4-yl)methyl)-1H-1,2,3-triazol-1-yl)-2-hydroxyacetic acid (C14-G2H); 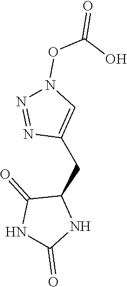 [SMILES: O=C1NC(=O)[C@@H](Cc2cn(OC(=O)O)nn2)N1]
(R)-4-((2,5-dioxoimidazolidin-4-yl)methyl)-1H-1,2,3-triazol-1-yl hydrogen carbonate (C14-G2I); and 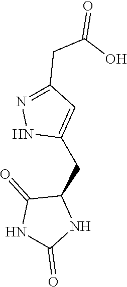 [SMILES: O=C(O)Cc1cc(C[C@H]2NC(=O)NC2=O)[nH]n1]
(R)-2-(5-((2,5-dioxoimidazolidin-4-yl)methyl)-1H-pyrazol-3-yl)acetic acid (C14-G2J).

15. The method of claim 12, wherein the bacterium is an antibiotic-resistant bacterium.

16. The method of claim 15, wherein the antibiotic resistance of the bacterium is derived from and/or involves formation of a biofilm or development of a stringent response by the bacterium.

17. The method of claim 12, wherein the bacterium is a gram-positive bacterium or a gram-negative bacterium.

18. The method of claim 12, wherein the bacterium is at least one of B. burgdorferi, E. coli, H. influenzae, N. gonorrhoeae, P. aeruginosa, S. epidermidis, S. pneumoniae, and S. aureus.

19. The method of claim 12, further comprises administering to the subject an antibiotic effective for killing or inhibiting growth of the bacterium.

20. The method of claim 12, wherein the subject is a mammal, optionally a human.

21. A composition, comprising:
at least one compound selected from:
(a) a compound of Formula I: 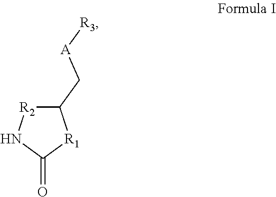
or a salt, solvate, tautomer, N-oxide, geometric isomer, stereoisomer thereof, and/or mixtures thereof, wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 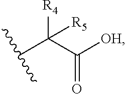
and
R₄ and R₅ are each independently H, halogen, C₁-C₆ alkyl, C₁-C₆ alkoxy, or —OH;
(b) 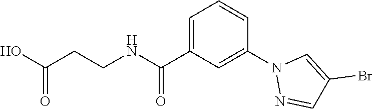 [SMILES: O=C(O)CCNC(=O)c1cccc(-n2cc(Br)cn2)c1]
3-(3-(4-bromo-1H-pyrazol-1-yl)benzamido)propanoic acid (C22), or a salt, solvate, tautomer, N-oxide, geometric isomer, and/or mixtures thereof;
and
an antibiotic.

22. The composition of claim 21, wherein in Formula I, A is 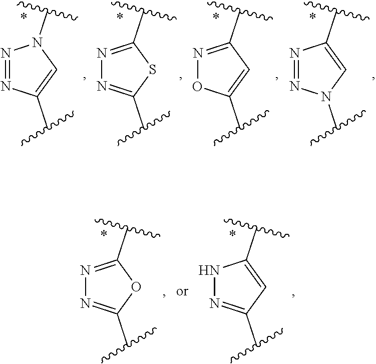 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]
wherein * is the bond to R₃ and wherein the CH in the five-membered heterocyclyl group of A (if present) is independently optionally substituted with at least one of C₁-C₆ alkyl, C₁-C₆ alkoxy, and halogen

23. The composition of claim 21, wherein the compound of Formula I is at least one selected from the group consisting of: 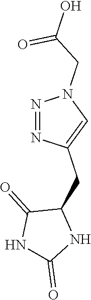 [SMILES: O=C(O)Cn1cc(C[C@H]2NC(=O)NC2=O)nn1]
(R)-2-(4-((2,5-dioxoimidazolidin-4-yl)methyl)-1H-1,2,3-triazol-1-yl)acetic acid (C14 or HEJ14); 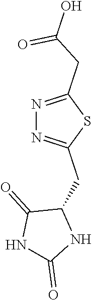 [SMILES: O=C(O)Cc1nnc(C[C@@H]2NC(=O)NC2=O)s1]
(S)-2-(5-((2,5-dioxoimidazolidin-4-yl)methyl)-1,3,4-thiadiazol-2-yl)acetic acid (C14-G2A); 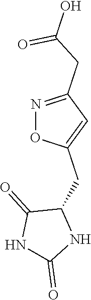 [SMILES: O=C(O)Cc1cc(C[C@@H]2NC(=O)NC2=O)on1]
(S)-2-(5-((2,5-dioxoimidazolidin-4-yl)methyl)isoxazol-3-yl)acetic acid (C14-G2B); 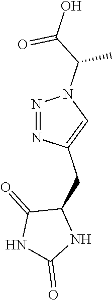 [SMILES: C[C@@H](C(=O)O)n1cc(C[C@H]2NC(=O)NC2=O)nn1]
(S)-2-(4-(((R)-2,5-dioxoimidazolidin-4-yl)methyl)-1H-1,2,3-triazol-1-yl)propanoic acid (C14-G2C); 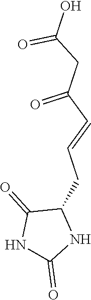 [SMILES: O=C(O)CC(=O)/C=C/C[C@@H]1NC(=O)NC1=O]
(S,E)-6-(2,5-dioxoimidazolidin-4-yl)-3-oxohex-4-enoic acid (C14-G2D); 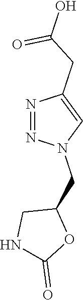 [SMILES: O=C(O)Cc1cn(C[C@@H]2CNC(=O)O2)nn1]
(S)-2-(1-((2-oxooxazolidin-5-yl)methyl)-1H-1,2,3-triazol-4-yl)acetic acid (C14-G2E); 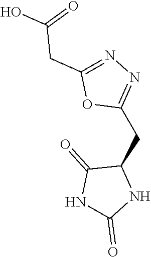 [SMILES: O=C(O)Cc1nnc(C[C@H]2NC(=O)NC2=O)o1]
(R)-2-(5-((2,5-dioxoimidazolidin-4-yl)methyl)-1,3,4-oxadiazol-2-yl)acetic acid (C14-G2F); 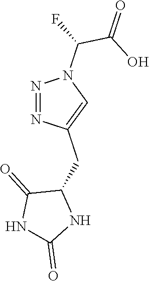 [SMILES: O=C1NC(=O)[C@H](Cc2cn([C@H](F)C(=O)O)nn2)N1]
(R)-2-(4-(((S)-2,5-dioxoimidazolidin-4-yl)methyl)-1H-1,2,3-triazol-1-yl)-2-fluoroacetic acid (C14-G2G); 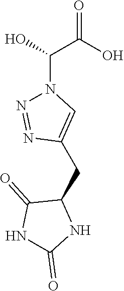 [SMILES: O=C1NC(=O)[C@@H](Cc2cn([C@H](O)C(=O)O)nn2)N1]
(R)-2-(4-(((R)-2,5-dioxoimidazolidin-4-yl)methyl)-1H-1,2,3-triazol-1-yl)-2-hydroxyacetic acid (C14-G2H); 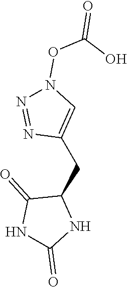 [SMILES: O=C1NC(=O)[C@@H](Cc2cn(OC(=O)O)nn2)N1]
(R)-4-((2,5-dioxoimidazolidin-4-yl)methyl)-1H-1,2,3-triazol-1-yl hydrogen carbonate (C14-G2I); and 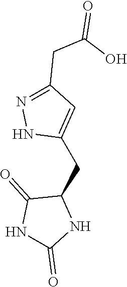 [SMILES: O=C(O)Cc1cc(C[C@H]2NC(=O)NC2=O)[nH]n1]
(R)-2-(5-((2,5-dioxoimidazolidin-4-yl)methyl)-1H-pyrazol-3-yl)acetic acid (C14-G2J).

24. The composition of claim 21, wherein the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

25. The composition of claim 21, further comprises a pharmaceutically acceptable carrier.

26. A kit, comprising:
at least one compound selected from:
(a) a compound of Formula I: 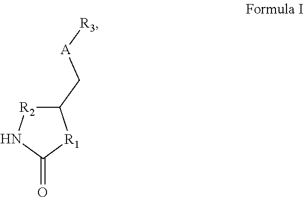
or a salt, solvate, tautomer, N-oxide, geometric isomer, stereoisomer thereof, and/or mixtures thereof, wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 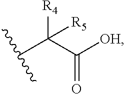
and
R₄ and R₅ are each independently H, halogen, C₁-C₆ alkyl, C₁-C₆ alkoxy, or —OH;
(b) 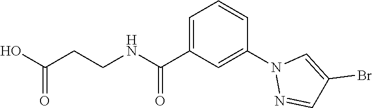 [SMILES: O=C(O)CCNC(=O)c1cccc(-n2cc(Br)cn2)c1]
3-(3-(4-bromo-1H-pyrazol-1-yl)benzamido)propanoic acid (C22), or a salt, solvate, tautomer, N-oxide, geometric isomer, and/or mixtures thereof;
and
an antibiotic.

27. The kit of claim 26, wherein in Formula I, A is 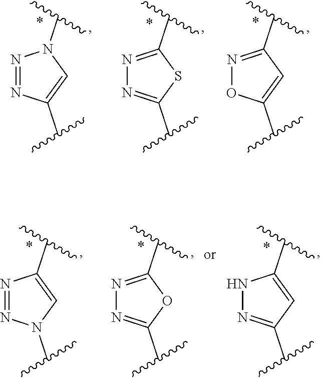 [SMILES: *c1cn(*)nn1 ; *c1nnc(*)s1 ; *c1cc(*)on1 ; *c1cn(*)nn1 ; *c1nnc(*)o1 ; *c1cc(*)[nH]n1]
wherein * is the bond to R₃ and wherein the CH in the five-membered heterocyclyl group of A (if present) is independently optionally substituted with at least one of C₁-C₆ alkyl, C₁-C₆ alkoxy, and halogen

28. The kit of claim 26, wherein the compound of Formula I is at least one selected from the group consisting of: 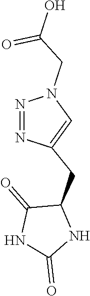 [SMILES: O=C(O)Cn1cc(C[C@H]2NC(=O)NC2=O)nn1]
(R)-2-(4-((2,5-dioxoimidazolidin-4-yl)methyl)-1H-1,2,3-triazol-1-yl)acetic acid (C14 or HEJ14); 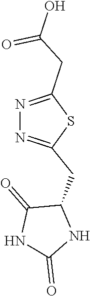 [SMILES: O=C(O)Cc1nnc(C[C@@H]2NC(=O)NC2=O)s1]
(S)-2-(5-((2,5-dioxoimidazolidin-4-yl)methyl)-1,3,4-thiadiazol-2-yl)acetic acid (C14-G2A); 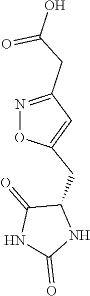 [SMILES: O=C(O)Cc1cc(C[C@@H]2NC(=O)NC2=O)on1]
(S)-2-(5-((2,5-dioxoimidazolidin-4-yl)methyl)isoxazol-3-yl)acetic acid (C14-G2B); 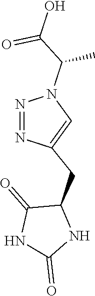 [SMILES: C[C@@H](C(=O)O)n1cc(C[C@H]2NC(=O)NC2=O)nn1]
(S)-2-(4-(((R)-2,5-dioxoimidazolidin-4-yl)methyl)-1H-1,2,3-triazol-1-yl)propanoic acid (C14-G2C); 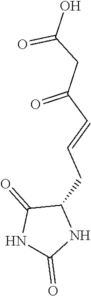 [SMILES: O=C(O)CC(=O)/C=C/C[C@@H]1NC(=O)NC1=O]
(S,E)-6-(2,5-dioxoimidazolidin-4-yl)-3-oxohex-4-enoic acid (C14-G2D); 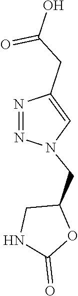 [SMILES: O=C(O)Cc1cn(C[C@@H]2CNC(=O)O2)nn1]
(S)-2-(1-((2-oxooxazolidin-5-yl)methyl)-1H-1,2,3-triazol-4-yl)acetic acid (C14-G2E); 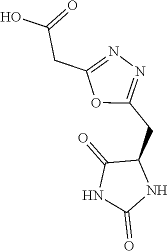 [SMILES: O=C(O)Cc1nnc(C[C@H]2NC(=O)NC2=O)o1]
(R)-2-(5-((2,5-dioxoimidazolidin-4-yl)methyl)-1,3,4-oxadiazol-2-yl)acetic acid (C14-G2F); 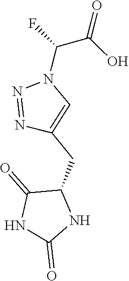 [SMILES: O=C1NC(=O)[C@H](Cc2cn([C@H](F)C(=O)O)nn2)N1]
(R)-2-(4-(((S)-2,5-dioxoimidazolidin-4-yl)methyl)-1H-1,2,3-triazol-1-yl)-2-fluoroacetic acid C14-G2G 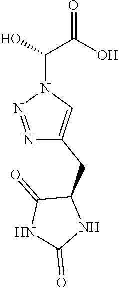 [SMILES: O=C1NC(=O)[C@@H](Cc2cn([C@H](O)C(=O)O)nn2)N1]
(R)-2-(4-(((R)-2,5-dioxoimidazolidin-4-yl)methyl)-1H-1,2,3-triazol-1-yl)-2-hydroxyacetic acid (C14-G2H); 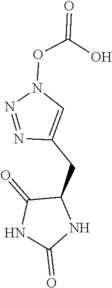 [SMILES: O=C1NC(=O)[C@@H](Cc2cn(OC(=O)O)nn2)N1]
(R)-4-((2,5-dioxoimidazolidin-4-yl)methyl)-1H-1,2,3-triazol-1-yl hydrogen carbonate (C14-G2I); and 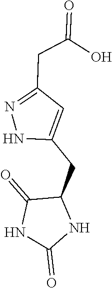 [SMILES: O=C(O)Cc1cc(C[C@H]2NC(=O)NC2=O)[nH]n1]
(R)-2-(5-((2,5-dioxoimidazolidin-4-yl)methyl)-1H-pyrazol-3-yl)acetic acid (C14-G2J).

29. The kit of claim 26, wherein the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

30. The kit of claim 26, further comprises a manual instructing that the compound and the antibiotic are to be administered to a subject infected with a bacterium, optionally an antibiotic-resistant bacterium.

31. A method of reducing acquisition of antibody resistance in a bacterium, the method comprising contacting the bacterium with at least one compound selected from:
(a) a compound of Formula I: 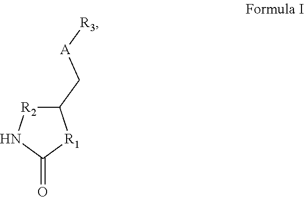
or a salt, solvate, tautomer, N-oxide, geometric isomer, stereoisomer thereof, and/or mixtures thereof, wherein:
R₁ is —NH— or —O—,
R₂ is —CH₂— or —C(O)—,
A is a five member aromatic heterocyclic ring or —CH═CH—COO—*, wherein * is the bond to R₃.
R₃ is —O—C(O)OH or 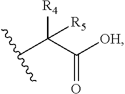
and
R₄ and R₅ are each independently H, halogen, C₁-C₆ alkyl, C₁-C₆ alkoxy, or —OH;
(b) 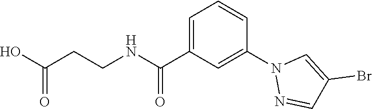 [SMILES: O=C(O)CCNC(=O)c1cccc(-n2cc(Br)cn2)c1]
3-(3-(4-bromo-1H-pyrazol-1-yl)benzamido)propanoic acid (C22), or a salt, solvate, tautomer, N-oxide, geometric isomer, and/or mixtures thereof.

32. The method of claim 31, wherein in Formula I, A is 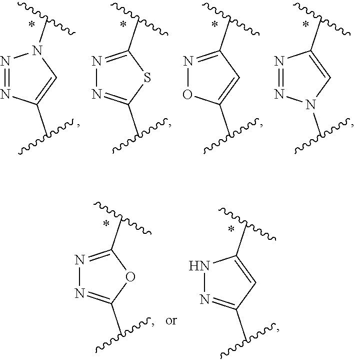
wherein * is the bond to R₃ and wherein the CH in the five-membered heterocyclyl group of A (if present) is independently optionally substituted with at least one of C₁-C₆ alkyl, C₁-C₆ alkoxy, and halogen.

33. The method of any one of claims 31-32, wherein the compound of Formula I is at least one selected from the group consisting of: 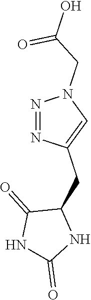 [SMILES: O=C(O)Cn1cc(C[C@H]2NC(=O)NC2=O)nn1]
(R)-2-(4-((2,5-dioxoimidazolidin-4-yl)methyl)-1H-1,2,3-triazol-1-yl)acetic acid (C14 or HEJ14); 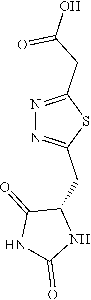 [SMILES: O=C(O)Cc1nnc(C[C@@H]2NC(=O)NC2=O)s1]
(S)-2-(5-((2,5-dioxoimidazolidin-4-yl)methyl)-1,3,4-thiadiazol-2-yl)acetic acid (C14-G2A); 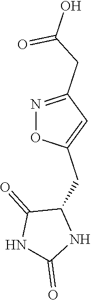 [SMILES: O=C(O)Cc1cc(C[C@@H]2NC(=O)NC2=O)on1]
(S)-2-(5-((2,5-dioxoimidazolidin-4-yl)methyl)isoxazol-3-yl)acetic acid (C14-G2B); 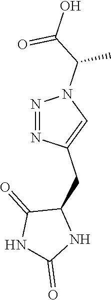 [SMILES: C[C@@H](C(=O)O)n1cc(C[C@H]2NC(=O)NC2=O)nn1]
(S)-2-(4-(((R)-2,5-dioxoimidazolidin-4-yl)methyl)-1H-1,2,3-triazol-1-yl)propanoic acid (C14-G2C); 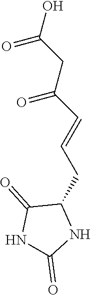 [SMILES: O=C(O)CC(=O)/C=C/C[C@@H]1NC(=O)NC1=O]
(S,E)-6-(2,5-dioxoimidazolidin-4-yl)-3-oxohex-4-enoic acid (C14-G2D); 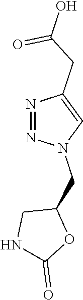 [SMILES: O=C(O)Cc1cn(C[C@@H]2CNC(=O)O2)nn1]
(S)-2-(1-((2-oxooxazolidin-5-yl)methyl)-1H-1,2,3-triazol-4-yl)acetic acid (C14-G2E); 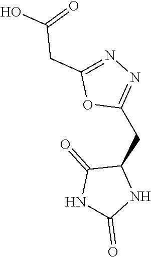 [SMILES: O=C(O)Cc1nnc(C[C@H]2NC(=O)NC2=O)o1]
(R)-2-(5-((2,5-dioxoimidazolidin-4-yl)methyl)-1,3,4-oxadiazol-2-yl)acetic acid (C14-G2F); 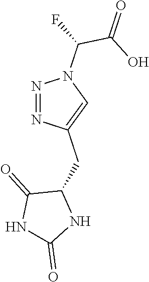 [SMILES: O=C1NC(=O)[C@H](Cc2cn([C@H](F)C(=O)O)nn2)N1]
(R)-2-(4-(((S)-2,5-dioxoimidazolidin-4-yl)methyl)-1H-1,2,3-triazol-1-yl)-2-fluoroacetic acid (C14-G2G); 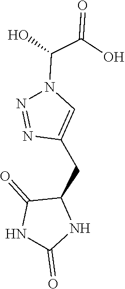 [SMILES: O=C1NC(=O)[C@@H](Cc2cn([C@H](O)C(=O)O)nn2)N1]
(R)-2-(4-(((R)-2,5-dioxoimidazolidin-4-yl)methyl)-1H-1,2,3-triazol-1-yl)-2-hydroxyacetic acid (C14-G2H); 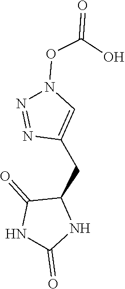 [SMILES: O=C1NC(=O)[C@@H](Cc2cn(OC(=O)O)nn2)N1]
(R)-4-((2,5-dioxoimidazolidin-4-yl)methyl)-1H-1,2,3-triazol-1-yl hydrogen carbonate (C14-G2I); and 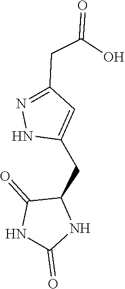 [SMILES: O=C(O)Cc1cc(C[C@H]2NC(=O)NC2=O)[nH]n1]
(R)-2-(5-((2,5-dioxoimidazolidin-4-yl)methyl)-1H-pyrazol-3-yl)acetic acid (C14-G2J).

34. The method of claim 31, further comprising contacting the bacterium with an antibiotic.

35. The method of claim 34, wherein the antibiotic comprises a penicillin class antibiotic, a tetracycline class antibiotic, a cephalosporin class antibiotic, a quinolone class antibiotic, a lincomycin class antibiotic, a macrolide antibiotic, a sulfonamide antibiotic, a glycopeptide antibiotic, an aminoglycoside antibiotic, a carbapenem antibiotic, or a combination thereof.

36. The method of claim 31, wherein the bacterium is at least one selected from the group consisting of B. burgdorferi, E. coli, H. influenzae, N. gonorrhoeae, P. aeruginosa, S. epidermidis, S. pneumoniae, and S. aureus.
